(19) United States
(12) Patent Application Publication
(10) Pub. No.: US 2022/0249087 A1
(43) Pub. Date: Aug. 11, 2022

(54) SUTURE PASSERS

(71) Applicants: SMITH & NEPHEW, INC., Memphis, TN (US); SMITH & NEPHEW ORTHOPAEDICS AG, Zug (CH); SMITH & NEPHEW ASIA PACIFIC PTE. LIMITED, Alice@Mediapolis (SG)

(72) Inventors: David R. Diduch, Charlottesville, VA (US); David Gregoire, Mission Viejo, CA (US); George W. White, Corona, CA (US)

(21) Appl. No.: 17/629,363

(22) PCT Filed: Jul. 22, 2020
(86) PCT No.: PCT/US20/43025

§ 371 Date: Jan. 21, 2022

Related U.S. Application Data
(60) Provisional application No. 62/877,277, filed on Jul. 22, 2019.

(51) Int. Cl. A61B 17/04; A61B 17/06; A61B 17/062

(52) U.S. Cl. CPC A61B 17/0482; A61B 17/0483; A61B 17/06004; A61B 2017/00862; A61B 17/062; A61B 2017/06042; A61B 2017/06095; A61B 17/06066

(57) ABSTRACT

Suture pulling devices are disclosed which are adapted to lock onto a target tissue by use of a pair of jaws and pull a strand of suture through the target tissue so that without opening the closed jaws, the strand of suture has been pulled through the tissues but maintained within the volume defined by the pair of jaws.

This Patent Application claims priority to U.S. Provisional Patent Application Ser. No. 62/877,277, filed Jul. 22, 2019, the content of which is hereby incorporated by reference herein in its entirety into this disclosure.

### TECHNICAL FIELD

The subject disclosure relates to suture passers. More particularly, the subject disclosure relates to suture passers for passing suture through a confined tissue sample.

### BACKGROUND

Arthroscopic surgery, laparoscopic surgery, and other less invasive surgical procedures require the suturing of soft tissue in an area not easily accessible due to the lack of space or direction of access to the soft tissue. Existing methods of suturing in such environments create a simple suture knot with only one suture pass through tissue due to the complexity of advancing suture in such tight access (confined) regions. A mattress suture configuration has been demonstrated as mechanically superior to single pass suturing and has a lower rate of pullout. However, passing a mattress suture (as well as a single pass suture) in less invasive procedures is exceedingly difficult and cumbersome using existing methods, which consist of rigid needles or snares that must be rotated through the soft tissue.

Various devices have been developed to address these issues. These devices are primarily suture pushers, and designed to push suture through a given volume of tissue using a needle that penetrates the tissue and extends beyond the confines of a given volume. For surgical procedures in small volume areas, such conventional devices may still penetrate surrounding tissue outside of the confines of the jaws. Adjacent structures can be injured or inadvertently pierced with the suture.

### SUMMARY OF THE SUBJECT DISCLOSURE

The present subject disclosure presents novel suture passer designs and methods for use which are intended to overcome the common shortcomings of the conventional suture passers. Furthermore, the present designs are intended to provide suture pulling devices which are adapted to lock onto a target tissue by use of a pair of jaws and pull a strand of suture through the target tissue so that without opening the closed jaws, the strand of suture has been pulled through the tissues but maintained within the volume defined by the pair of jaws.

In one exemplary embodiment, the present subject matter is a suture pulling device. The device includes a shaft having a proximal end, a distal end, and a longitudinal axis extending therebetween; a first jaw member and a second jaw member connected to the shaft distal end, the first jaw member moveable relative to the second jaw member; a tissue penetrating member positioned substantially within and slidable through the first jaw, the tissue penetrating member including a transverse opening configured to selectively carry a suture; a suture accommodating trough positioned within the second jaw and adapted to hold a strand of suture; wherein the tissue penetrating member is slidable between a first and a second position, wherein in the first position the tissue penetrating member is recessed within the first jaw member and wherein in the second position the tissue penetrating member extends through a volume defined within the first jaw and the second jaw so that the transverse opening of the tissue penetrating member engages with the strand of suture positioned in the suture accommodating trough of the second jaw.

In another exemplary embodiment, the present subject matter is a suture puling device. The device includes a shaft having a proximal end, a distal end, and a longitudinal axis extending therebetween; a first jaw member and a second jaw member connected to the shaft distal end, the first jaw member moveable relative to the second jaw member; a tissue penetrating member positioned substantially within and slidable through the first jaw, the tissue penetrating member including a transverse opening configured to selectively carry a suture; an asymmetric V-shaped suture accommodating trough positioned within the second jaw and adapted to hold a strand of suture, wherein the suture accommodating trough is positioned so that two ends of the strand of suture project from a side portion of the second jaw; wherein the tissue penetrating member is slidable between a first and a second position, wherein in the first position the tissue penetrating member is recessed within the first jaw member and wherein in the second position the tissue penetrating member extends through a volume defined within the first jaw and the second jaw so that the transverse opening of the tissue penetrating member engages with the strand of suture positioned in the suture accommodating trough of the second jaw.

In yet another exemplary embodiment, the present subject matter is a suture puling device. The device includes a shaft having a proximal end, a distal end, and a longitudinal axis extending therebetween; a first jaw member and a second jaw member connected to the shaft distal end, the first jaw member moveable relative to the second jaw member; a tissue penetrating member positioned substantially within and slidable through the first jaw, the tissue penetrating member including a transverse opening configured to selectively carry a suture; an asymmetric V-shaped suture accommodating trough positioned within the second jaw and adapted to hold a strand of suture, wherein the suture accommodating trough is positioned so that two ends of the strand of suture project from opposite sides of the second jaw; wherein the tissue penetrating member is slidable between a first and a second position, wherein in the first position the tissue penetrating member is recessed within the first jaw member and wherein in the second position the tissue penetrating member extends through a volume defined within the first jaw and the second jaw so that the transverse opening of the tissue penetrating member engages with the strand of suture positioned in the suture accommodating trough of the second jaw.

### BRIEF DESCRIPTION OF THE DRAWINGS

Various exemplary embodiments of this disclosure will be described in detail, wherein like reference numerals refer to identical or similar components or steps, with reference to the following figures, wherein:

FIG. 1A illustrates a top view of a suture passing device, according to an exemplary embodiment of the present subject disclosure.

FIG. 1B illustrates a side view of a suture passing device, according to an exemplary embodiment of the present subject disclosure.

FIG. 1C illustrates a bottom view of a suture passing device, according to an exemplary embodiment of the present subject disclosure.

FIG. 1D illustrates a front view of a suture passing device, according to an exemplary embodiment of the present subject disclosure.

FIG. 2A illustrates a front perspective view with partial cross section of a suture passing device, according to an exemplary embodiment of the present subject disclosure.

FIG. 2B illustrates a top view of a bottom jaw of a suture passing device with a top jaw removed, according to an exemplary embodiment of the present subject disclosure.

FIG. 3 illustrates a front perspective view of a suture passing device with a suture in position, according to an exemplary embodiment of the present subject disclosure.

FIG. 4A illustrates a cross sectional view of a suture engaging portion of a needle before engaging a strand of suture in a suture passing device, according to an exemplary embodiment of the present subject disclosure.

FIG. 4B illustrates a cross sectional view of a suture engaging portion of a needle engaging a strand of suture in a suture passing device, according to an exemplary embodiment of the present subject disclosure.

FIG. 4C illustrates a cross sectional view of a suture engaging portion of a needle after engaging a strand of suture in a suture passing device, according to an exemplary embodiment of the present subject disclosure.

FIG. 4D illustrates a front perspective view of a needle, according to an exemplary embodiment of the present subject disclosure.

FIG. 5A illustrates a front perspective view of a suture passing device with a strand of suture being engaged by a suture engaging portion of a needle, according to an exemplary embodiment of the present subject disclosure.

FIG. 5B illustrates a front perspective view of a suture passing device with a strand of suture being pulled up by a suture engaging portion of a needle, according to an exemplary embodiment of the present subject disclosure.

FIG. 6A illustrates a top view of a suture passing device, according to an exemplary embodiment of the present subject disclosure.

FIG. 6B illustrates a side view of a suture passing device, according to an exemplary embodiment of the present subject disclosure.

FIG. 6C illustrates a bottom view of a suture passing device, according to an exemplary embodiment of the present subject disclosure.

FIG. 6D illustrates a front view of a suture passing device, according to an exemplary embodiment of the present subject disclosure.

FIG. 7A illustrates a front perspective view with partial cross section of a suture passing device, according to an exemplary embodiment of the present subject disclosure.

FIG. 7B illustrates a top view of a bottom jaw of a suture passing device with a top jaw removed, according to an exemplary embodiment of the present subject disclosure.

FIG. 8 illustrates a front perspective view of a suture passing device with a suture in position, according to an exemplary embodiment of the present subject disclosure.

FIG. 9A illustrates a cross sectional view of a suture engaging portion of a needle before engaging a strand of suture in a suture passing device, according to an exemplary embodiment of the present subject disclosure.

FIG. 9B illustrates a cross sectional view of a suture engaging portion of a needle engaging a strand of suture in a suture passing device, according to an exemplary embodiment of the present subject disclosure.

FIG. 9C illustrates a cross sectional view of a suture engaging portion of a needle after engaging a strand of suture in a suture passing device, according to an exemplary embodiment of the present subject disclosure.

FIG. 9D illustrates a front perspective view of a needle, according to an exemplary embodiment of the present subject disclosure.

FIG. 10A illustrates a front perspective view of a suture passing device with a strand of suture being engaged by a suture engaging portion of a needle, according to an exemplary embodiment of the present subject disclosure.

FIG. 10B illustrates a front perspective view of a suture passing device with a strand of suture being pulled up by a suture engaging portion of a needle, according to an exemplary embodiment of the present subject disclosure.

FIG. 11A illustrates a top view of a suture passing device, according to an exemplary embodiment of the present subject disclosure.

FIG. 11B illustrates a side view of a suture passing device, according to an exemplary embodiment of the present subject disclosure.

FIG. 11C illustrates a bottom view of a suture passing device, according to an exemplary embodiment of the present subject disclosure.

FIG. 11D illustrates a front view of a suture passing device, according to an exemplary embodiment of the present subject disclosure.

FIG. 12A illustrates a front perspective view with partial cross section of a suture passing device, according to an exemplary embodiment of the present subject disclosure.

FIG. 12B illustrates a top view of a bottom jaw of a suture passing device with a top jaw removed, according to an exemplary embodiment of the present subject disclosure.

FIG. 13 illustrates a front perspective view of a suture passing device with a suture in position, according to an exemplary embodiment of the present subject disclosure.

FIG. 14A illustrates a cross sectional view of a suture engaging portion of a needle before engaging a strand of suture in a suture passing device, according to an exemplary embodiment of the present subject disclosure.

FIG. 14B illustrates a cross sectional view of a suture engaging portion of a needle engaging a strand of suture in a suture passing device, according to an exemplary embodiment of the present subject disclosure.

FIG. 14C illustrates a cross sectional view of a suture engaging portion of a needle after engaging a strand of suture in a suture passing device, according to an exemplary embodiment of the present subject disclosure.

FIG. 14D illustrates a front perspective view of a needle, according to an exemplary embodiment of the present subject disclosure.

FIG. 15A illustrates a front perspective view of a suture passing device with a strand of suture being engaged by a suture engaging portion of a needle, according to an exemplary embodiment of the present subject disclosure.

FIG. 15B illustrates a front perspective view of a suture passing device with a strand of suture being pulled up by a suture engaging portion of a needle, according to an exemplary embodiment of the present subject disclosure.

FIG. 16A illustrates a top view of a suture passing device, according to an exemplary embodiment of the present subject disclosure.

FIG. 16B illustrates a side view of a suture passing device, according to an exemplary embodiment of the present subject disclosure.

FIG. 16C illustrates a bottom view of a suture passing device, according to an exemplary embodiment of the present subject disclosure.

FIG. 16D illustrates a front view of a suture passing device, according to an exemplary embodiment of the present subject disclosure.

FIG. 17A illustrates a front perspective view with partial cross section of a suture passing device, according to an exemplary embodiment of the present subject disclosure.

FIG. 17B illustrates a top view of a bottom jaw of a suture passing device with a top jaw removed, according to an exemplary embodiment of the present subject disclosure.

FIG. 18 illustrates a front perspective view of a suture passing device with a suture in position, according to an exemplary embodiment of the present subject disclosure.

FIG. 19A illustrates a cross sectional view of a suture engaging portion of a needle before engaging a strand of suture in a suture passing device, according to an exemplary embodiment of the present subject disclosure.

FIG. 19B illustrates a cross sectional view of a suture engaging portion of a needle engaging a strand of suture in a suture passing device, according to an exemplary embodiment of the present subject disclosure.

FIG. 19C illustrates a cross sectional view of a suture engaging portion of a needle after engaging a strand of suture in a suture passing device, according to an exemplary embodiment of the present subject disclosure.

FIG. 19D illustrates a front perspective view of a needle, according to an exemplary embodiment of the present subject disclosure.

FIG. 20A illustrates a front perspective view of a suture passing device with a strand of suture being engaged by a suture engaging portion of a needle, according to an exemplary embodiment of the present subject disclosure.

FIG. 20B illustrates a front perspective view of a suture passing device with a strand of suture being pulled up by a suture engaging portion of a needle, according to an exemplary embodiment of the present subject disclosure.

FIG. 21A illustrates a top view of a suture passing device, according to an exemplary embodiment of the present subject disclosure.

FIG. 21B illustrates a side view of a suture passing device, according to an exemplary embodiment of the present subject disclosure.

FIG. 21C illustrates a bottom view of a suture passing device, according to an exemplary embodiment of the present subject disclosure.

FIG. 21D illustrates a front view of a suture passing device, according to an exemplary embodiment of the present subject disclosure.

FIG. 22A illustrates a front perspective view with partial cross section of a suture passing device, according to an exemplary embodiment of the present subject disclosure.

FIG. 22B illustrates a top view of a bottom jaw of a suture passing device with a top jaw removed, according to an exemplary embodiment of the present subject disclosure.

FIG. 22C illustrates a side view of suture capturing mechanisms, according to an exemplary embodiment of the present subject disclosure.

FIG. 23 illustrates a front perspective view of a suture passing device with a suture in position, according to an exemplary embodiment of the present subject disclosure.

FIG. 24A illustrates a cross sectional view of a suture engaging portion of a needle before engaging a strand of suture in a suture passing device, according to an exemplary embodiment of the present subject disclosure.

FIG. 24B illustrates a cross sectional view of a suture engaging portion of a needle engaging a strand of suture in a suture passing device, according to an exemplary embodiment of the present subject disclosure.

FIG. 24C illustrates a cross sectional view of a suture engaging portion of a needle after engaging a strand of suture in a suture passing device, according to an exemplary embodiment of the present subject disclosure.

FIG. 24D illustrates a front perspective view of a needle, according to an exemplary embodiment of the present subject disclosure.

FIG. 25A illustrates a front perspective view of a suture passing device with a strand of suture being engaged by a suture engaging portion of a needle, according to an exemplary embodiment of the present subject disclosure.

FIG. 25B illustrates a front perspective view of a suture passing device with a strand of suture being pulled up by a suture engaging portion of a needle, according to an exemplary embodiment of the present subject disclosure.

FIG. 26A illustrates a top view of an angled suture passing device, according to an exemplary embodiment of the present subject disclosure.

FIG. 26B illustrates a side view of an angled suture passing device, according to an exemplary embodiment of the present subject disclosure.

FIG. 26C illustrates a bottom view of an angled suture passing device, according to an exemplary embodiment of the present subject disclosure.

FIG. 26D illustrates a front view of an angled suture passing device, according to an exemplary embodiment of the present subject disclosure.

### DETAILED DESCRIPTION

Particular embodiments of the present subject disclosure will now be described in greater detail with reference to the figures.

The subject disclosure is described with reference to the drawings, wherein like reference numerals are used to refer to like elements throughout. In the following description, for purposes of explanation, numerous specific details are set forth in order to provide a thorough understanding of the present disclosure. It may be evident, however, that the present disclosure may be practiced without these specific details.

As employed in this specification and annexed drawings, the term “or” is intended to mean an inclusive “or” rather than an exclusive “or.” Moreover, articles “a” and “an” as used in the subject specification and annexed drawings should generally be construed to mean “one or more” unless specified otherwise or clear from context to be directed to a singular form.

Various exemplary embodiments are presented in this disclosure. The function and movement of the jaws of a suture device, as well as various types of superelastic or nitinol needles are based substantially on the functioning of other similar suture devices, such as, for example, that of Diduch et al., U.S. Pat. No. 9,393,009, which is incorporated by reference herein in its entirety into this disclosure, and will not be described in any detail here.

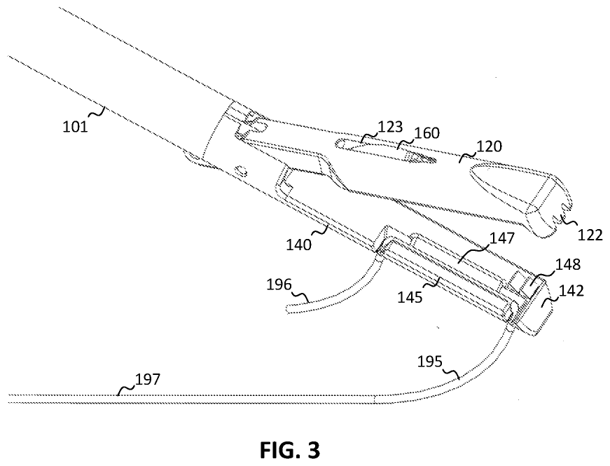

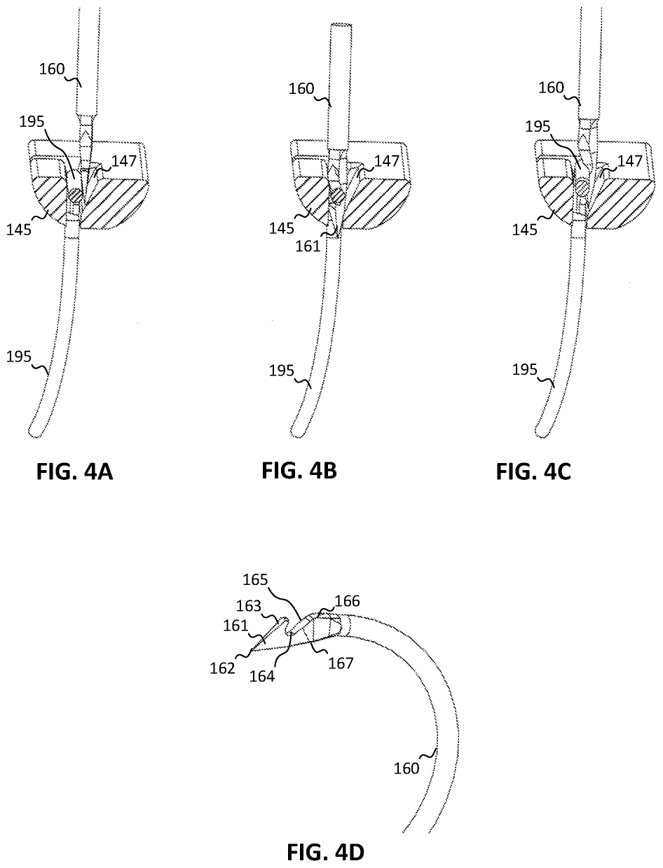

The present subject disclosure relates to the puling of suture through tissue as opposed to the pushing of suture through tissue, as used in many conventional device and techniques. The embodiments presented in this disclosure relate to the pulling of a strand of suture through tissue, which is in a defined space limited by the two jaws. The needle tip does not substantially protrude outside of the jaws and the suture is confined within the space within the jaws. This provides a very controlled pull of suture within a very defined volume of tissue without extending beyond the volume created by the two jaws. As shown in, for example, FIG. 3 and elsewhere, a portion of the needle (such as its belly labeled 160) may bow and partially extend beyond the confines of the jaws. But this portion of the needle is not sharp and has little to no effect on the tissue outside of the jaws. As shown in, for example, FIG. 4B and elsewhere, the tip 161 of the needle may protrude slightly outside the outer edge of the jaws in order to allow the capture of the suture, but the amount protruded out is minimal and is not intended to interact with the surrounding tissue.

One of the many unique features and attributes of the present subject disclosure is the needle positioned within the moving jaw. For the purpose of retrieving (rather than advancing) suture through tissue, the needle goes past the suture into a trough and then moves or twists in such a way that the crochet hook can then engage and capture the suture. This requires the lower jaw containing the trough to be very stable as the needle is pushed to the bottom of this trough, causing it to deflect or twist. If the needle were going into a moving, less rigid upper jaw instead, the path could be altered causing the suture to be missed. Also, the movable upper jaw allows that jaw angle to be adjusted to sit directly on the surface of the tissue being grasped such that the needle always enters at the optimal angle and travels a more predictable path through the tissue. This helps the needle to find the target of the suture and the trough on the far side of the tissue. The larger the bite of tissue, the more important it becomes to control the needle path.

The present subject disclosure presents a number of exemplary embodiments, which are not limited to the specific components presented in each embodiment, but whose components may be interchanged with the components of other exemplary embodiments. Each exemplary embodiment describes the shape of a needle tip with specific upper and lower edges/angles/etc. It should be noted that any of these components with specific features may be interchanged with other comparable components with other specific features in other embodiments, and the principles of function remain the same. For example, in some embodiments, the needle tip could have the wider, beveled edge opposite the crochet hook, with the crochet hook on the thin, sharp edge. This may help prevent tip breakage. Alternatively, in other embodiments, the thicker, beveled edge could be on the crochet hook side to enhance suture engagement. The needle could also be symmetrically tapered in shape with the crochet hook notch on one side for those applications in which the trough shape is causing the needle to change paths in order to slide under and engage the suture. One of ordinary skill in the art, after consideration of the present disclosure, would appreciate the interchangeability of the specific components of one exemplary embodiment with the specific components of another exemplary embodiment, without departing from the scope of the present disclosure.

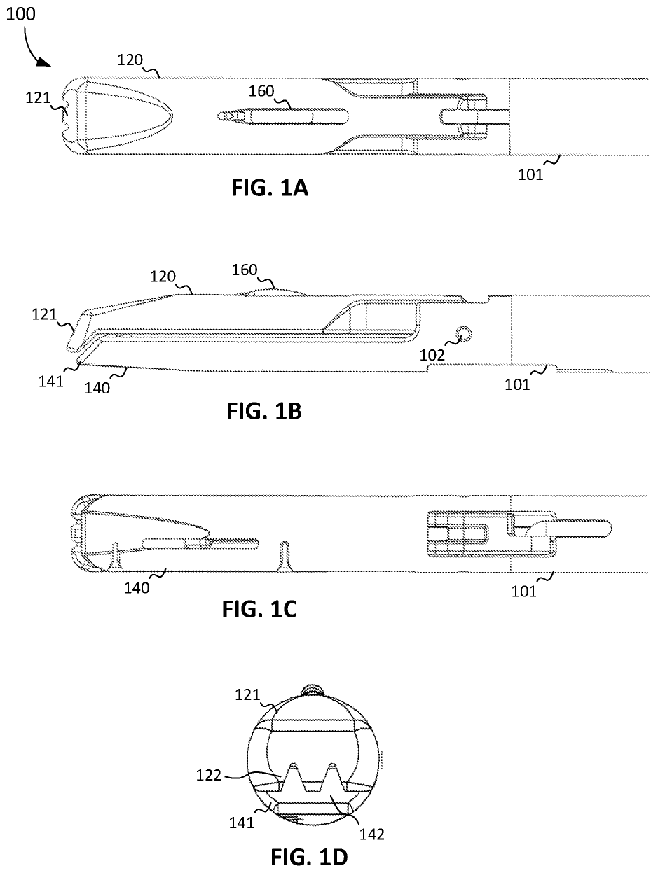

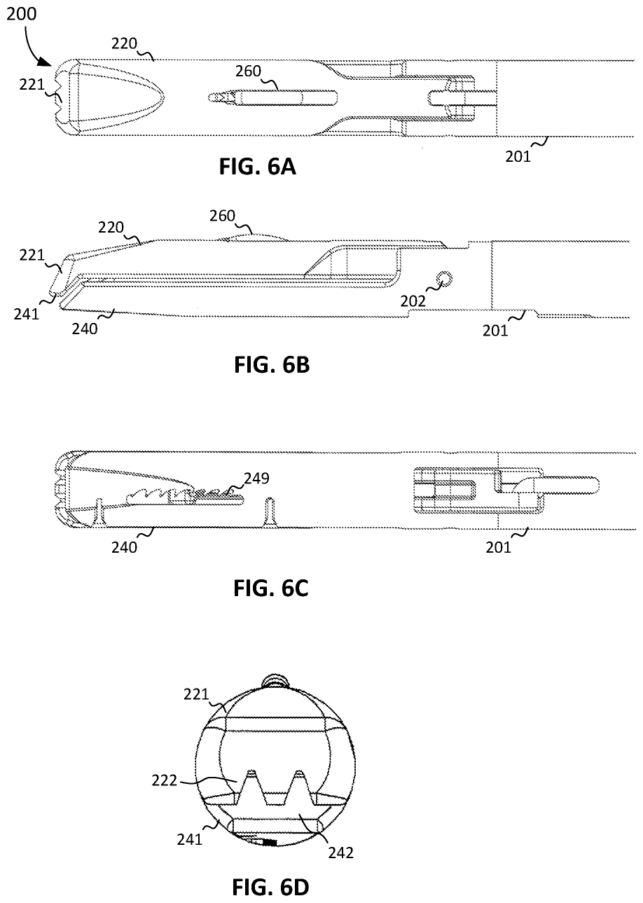

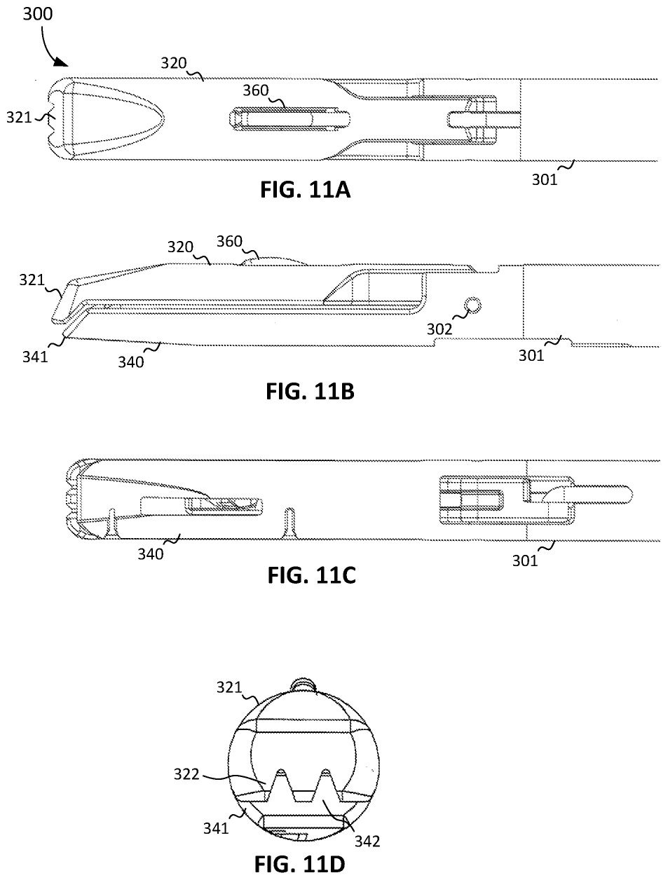

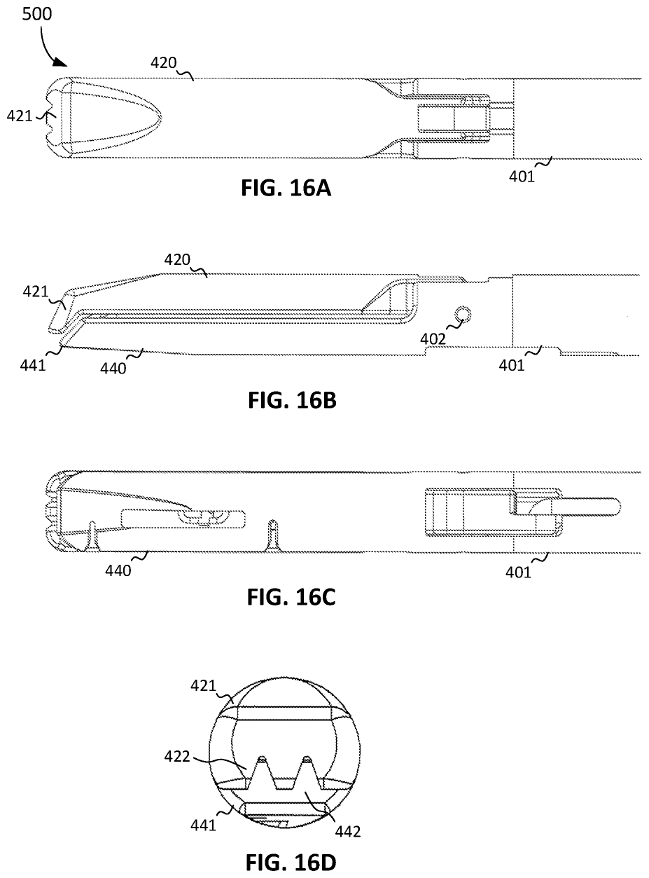

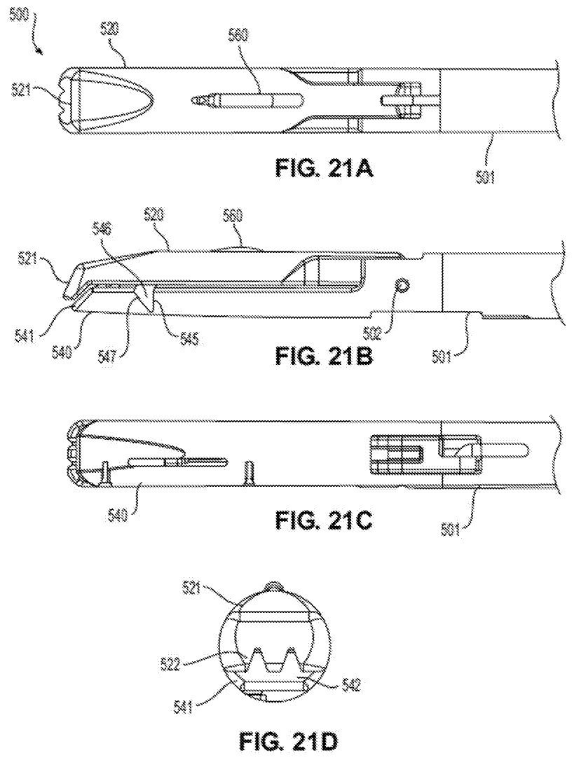

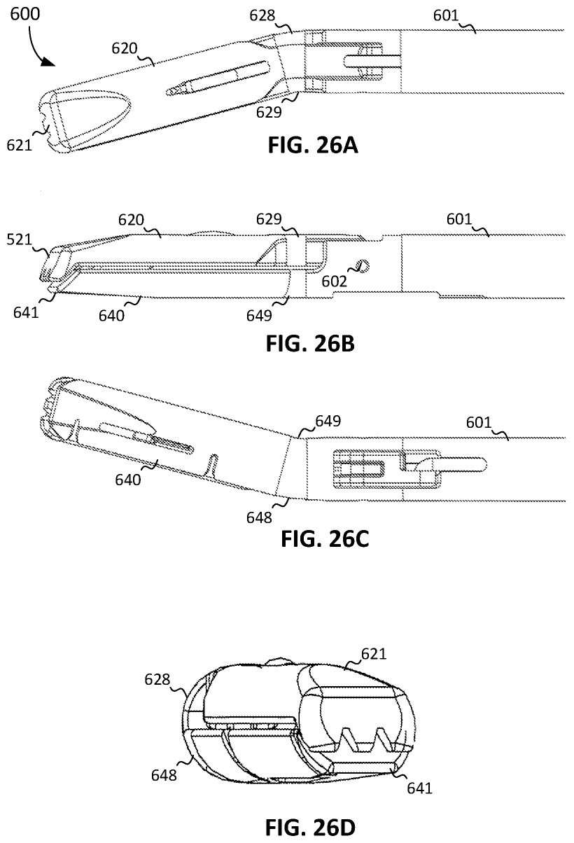

A first exemplary embodiment, presented in FIGS. 1-5, includes a pre-curved needle that twists upon contact with a sloped surface to pick up a suture. A second exemplary embodiment, presented in FIGS. 6-10, includes a pre-curved needle that twists upon contact with a plurality of different angled grooves or twist surfaces to pick up a suture. A third exemplary embodiment, presented in FIGS. 11-15, includes a pre-curved needle that gets directed by a sloped surface to pick up a suture without further twisting of the needle. A fourth exemplary embodiment, presented in FIGS. 16-20, includes a straight needle that gets directed by a sloped surface to pick up a suture. A fifth exemplary embodiment, presented in FIGS. 21-25, includes a pre-curved needle with the suture engaging portion positioned on the inner curve that gets directed by a sloped surface to pick up a suture from a transverse trough without further twisting of the needle. A sixth exemplary embodiment, presented in FIGS. 26A-D, includes an angled jaw configuration which may be used for any of the four embodiments presented in FIGS. 1-25. The example of the angle shown in FIGS. 26A-D is an angle right, but any other angle, such as up, left, right, and combination of angles, such as up-right, up-left, etc., may also be used, and within the purview of the present disclosure.

FIGS. 1-5 illustrate a suture passer device according to a first exemplary embodiment of the present subject disclosure. The device 100 includes an elongated shaft body 101 connecting to a pair of jaws, including an upper jaw 120 and a lower jaw 140. The upper jaw 120 and lower jaw 140 move relative to each other about a rotation point 102, which may be a hinge or other similar mechanism. Upper jaw 120 has a distal end 121 and lower jaw 140 has a distal end 141. The upper jaw distal end 121 is adapted to fit over the lower jaw distal end 141. However, the opposite is also possible in that the lower jaw distal end 141 may be adapted to fit over the upper jaw distal end 121.

Upper jaw distal end 121 may have one or more external teeth 122 which serve to grab unto tissue held within the pair of jaws 120/140. The lower jaw distal end 141 may have a generally flat and inclined surface to accommodate an opposite side of the tissue which is being held by the external teeth 122. Optionally, external teeth 122 may be present on both the upper jaw distal end 121 and lower jaw distal end 141. Also possible is that the lower jaw distal end 141 has external teeth 122, or neither jaw 120/140 has any teeth at all.

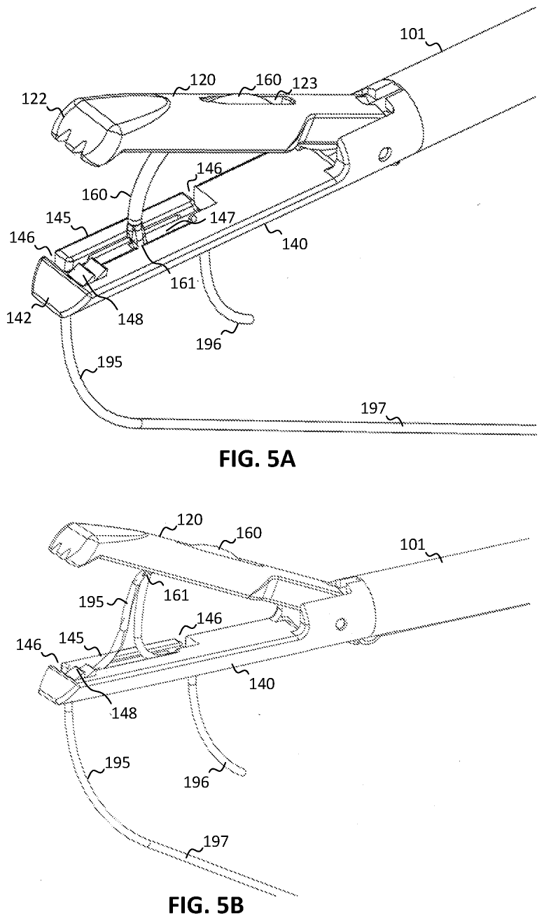

As shown in FIGS. 3, 5A-5B, lower jaw 140 may have internal teeth 148 which serve to help retain tissue held between the upper jaw 120 and lower jaw 140. Internal teeth 148 have an inclined surface facing the distal end 141 of the lower jaw 140 and a relatively vertical wall facing away from the distal end 141 of the lower jaw 140. This shape is akin to backward facing shark teeth in retaining material held within the jaws 120/140 and minimizing or preventing slip of tissue material once the jaws 120/140 are gripping the material. Thus, the external teeth 122 on the upper jaw 120 and the internal teeth 148 on the lower jaw 140 may work together to firmly hold tissue material within the mouth of the jaws while the suture pulling mechanism is used to pull suture through the tissue material, as will be described in more detail below.

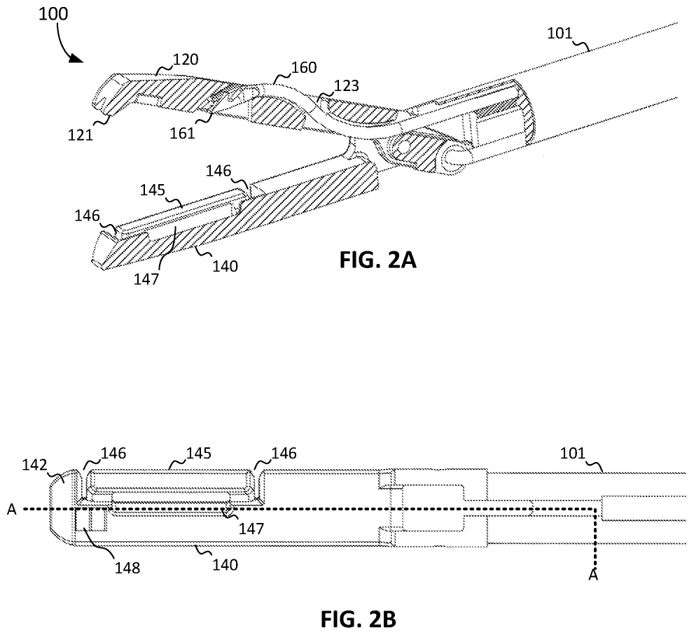

FIG. 2A shows a perspective view with partial cross sectional cut along line A-A shown in the top view illustration of FIG. 2B. It should be noted that FIG. 2B does not have the top jaw portion 120 while FIG. 2A does include the top jaw portion 120. As shown in FIG. 2A, top jaw 120 includes an opening 123 which allows a needle 160 body to traverse therethrough. The needle 160 includes a substantial portion which is housed within the elongated shaft 101 of the device 100, and terminates at a distally positioned suture engagement end 161.

Also included is a V-shaped asymmetric trough 146 formed in the bottom jaw 140 and used to accommodate a suture before it is pulled through tissue. The top down view of the trough 146 may be any shape, including oval, rectangular, circular, etc., as long as it performs the function as shown and described herein. In this particular example, the trough 146 includes three portions, including an elongated central portion which extends along the longitudinal axis of the shaft 101, and the lower jaw 140, and two connecting portions which are perpendicular to the elongated portion. The elongated central portion of the asymmetric trough 146 also includes an opening through which the tip 162 of the needle 160 projects therethrough in order to capture the suture 195, as will be described in more detail below. The trough 146 includes two connecting portions which extend out a side of the lower portion 140, as shown in FIG. 2B. A central wall 145 serves to form an internal wall of the trough 146. A suture is routed through the three portions of the trough 146, as will be described in more detail below.

FIG. 3 shows a strand of suture 195 placed within the trough 146 of bottom jaw 140. The strand of suture 195 is wrapped around the vertical wall 145 and pulled so that the two ends 196/197 of the suture 195 extend out beyond the edges of lower jaw 140. A proximal end 196 of the suture 195 is positioned closer to the shaft 101, and a distal end 197 is positioned further from the shaft 101 and closer to the end 141 of the lower jaw 140. FIG. 3 illustrates the position of the suture 195 within the trough 146 of the device 100 directly before use of the device 100. It should be noted that although the trough 146/147 in the exemplary embodiment has been shown in the figures as having a specific configuration, the same technique may be used if the trough holding the suture was oriented 90 degrees (or other angle) to what is shown in the drawings. In other words, instead of the central portion of the trough 146 being longitudinal across the lower jaw 140, it is transverse.

FIG. 4D shows a detailed view of a needle 160 used in the exemplary embodiment presented in FIGS. 1-5 of the present disclosure. Needle 160 includes an elongated shaft with a suture engaging portion 161 at its distal end. The suture engaging portion 161 includes a pointed or sharpened tip 162 and two sides, a top side 166 and a bottom side 167. A straight edge bottom side 167 includes a tapered edge. A top side 166 has a flattened, square edge. Three edges are formed between the top side 166 and bottom side 167. The top side 166 has two edges and the bottom side 167 has one edge. The angle formed between the bottom side 167 edge and top side 166 edges may be, for example, between 10 and 60 degrees. In particular, the angle formed may be between 30-45 degrees. One exemplary angle edge is 30 degrees. Another exemplary angle edge is 45 degrees. The angled edge gives the suture engaging portion 161 a twist, which is used to engage a strand of suture, as will be described in detail below.

The suture engaging portion 161 includes a hook portion 163/164/165 in the shape of a crochet hook. The hook portion 163/164/165 includes a first edge 163, a concave trough 164, and a top sloped edge 165 that leads into the top side 166 of the suture engaging portion 161. The concave trough 164 accommodates a strand of suture 195 so it should be sized and textured to hold various sized sutures without damaging them.

FIGS. 4A-4C show the interaction of the suture engaging portion 161 of the needle 160 with the trough 146. In this embodiment, the actual shape of the needle 161 and different orientation of the bevels cause the needle tip to twist as it advances into the trough 147 as described. As shown in FIG. 4A, as the suture engaging portion 161 enters the trough 147, the hook portion 163/164/165 makes contact with the sloped sidewall of the concave trough 147 and the strand of suture 195. The tapered shape of the suture engaging portion 161 adapts it to twist so that the needle tip 162 angles underneath the strand of suture 195, as shown in FIG. 4B. The strand of suture 195 is then positioned within the concave trough 164. As shown in FIG. 4C, the strand 195 is positioned within the concave trough 164 as the needle tip 162 is being pulled back up so that the strand of suture 195 is fully controlled by suture engaging portion 161. The external shape of the suture engaging portion 161 allows an angled twist of the suture engaging portion 161 as it comes into contact with the angled wall of the asymmetric V-shaped trough 147, thereby positioning the hook portion 163/164/165 underneath the strand of suture 195 so that the strand of suture 195 is cradled and pulled up, as will be described in more detail below.

FIG. 5A shows a perspective view of the device 100 as the suture engaging portion 161 is engaged with the strand of suture 195, the detail of which is shown in FIG. 4C. Once the concave trough 164 of the needle tip 161 has engaged the strand of suture 195, the needle shaft 160 is pulled back so that the needle 160 retracts back into its original starting position, as shown in FIG. 5B. The movement of the suture engaging portion 161 between FIG. 5A and FIG. 5B results in the pulling of the strand of suture 195 through any tissue material that is held in place between the upper jaw 120 and lower jaw 140.

In use, the device 100 starts in a closed jaw position, such as those shown in FIGS. 1A-1D. Once a target tissue is desired to be repaired, the jaws 120/140 are opened, revealing an internal V-shaped asymmetric trough 146. A strand of suture 195 is then positioned within the trough 146 so that the ends 196/197 of the suture 195 extend out from the edge of the lower jaw 140, as shown in FIG. 3. The open jaws 120/140 are then positioned about a target tissue which needs to be repaired. The external teeth 122 and internal teeth 148 serve to maintain the position of the target tissue with respect to the jaws 120/140. Once the target tissue (not shown) is positioned and held within the open jaws 120/140, as shown in FIG. 3, the needle 160 is then pushed through the target tissue. The suture engaging portion 161 is pushed through the tissue until it comes into contact with the sloped wall 147 of the bottom jaw 140, as shown in FIG. 4A. The suture engaging portion 161 then twists and extends underneath the strand of suture 195 that was positioned in trough 146 until the hook portion 163/164/165 slips underneath the strand of suture 195, as shown in FIGS. 4B and 5A. Once the strand of suture 195 is cradled within the suture trough 14664, the needle 160 is pulled back up through the target tissue, bringing with it the strand of suture 195, as shown in FIGS. 4C and 5B. At this point, the strand of suture has been pulled through the tissue and may be used in creating various stitches as needed.

FIGS. 6-10 illustrate a suture passer device according to a second exemplary embodiment of the present subject disclosure. The device 200 includes an elongated shaft body 201 connecting to a pair of jaws, including an upper jaw 220 and a lower jaw 240. The upper jaw 220 and lower jaw 240 move relative to each other about a rotation point 202, which may be a hinge or other similar mechanism. Upper jaw 220 has a distal end 221 and lower jaw 240 has a distal end 241. The upper jaw distal end 221 is adapted to fit over the lower jaw distal end 241. However, the opposite is also possible in that the lower jaw distal end 241 may be adapted to fit over the upper jaw distal end 221.

Upper jaw distal end 221 may have one or more external teeth 222 which serve to grab unto tissue held within the pair of jaws 220/240. The lower jaw distal end 241 may have a generally flat and inclined surface to accommodate an opposite side of the tissue which is being held by the external teeth 222. Optionally, external teeth 222 may be present on both the upper jaw distal end 221 and lower jaw distal end 241. Also possible is that the lower jaw distal end 241 has external teeth 222, or neither jaw 220/240 has any teeth at all.

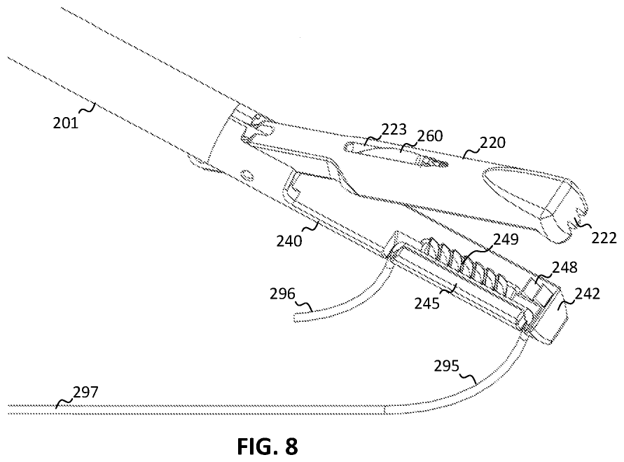

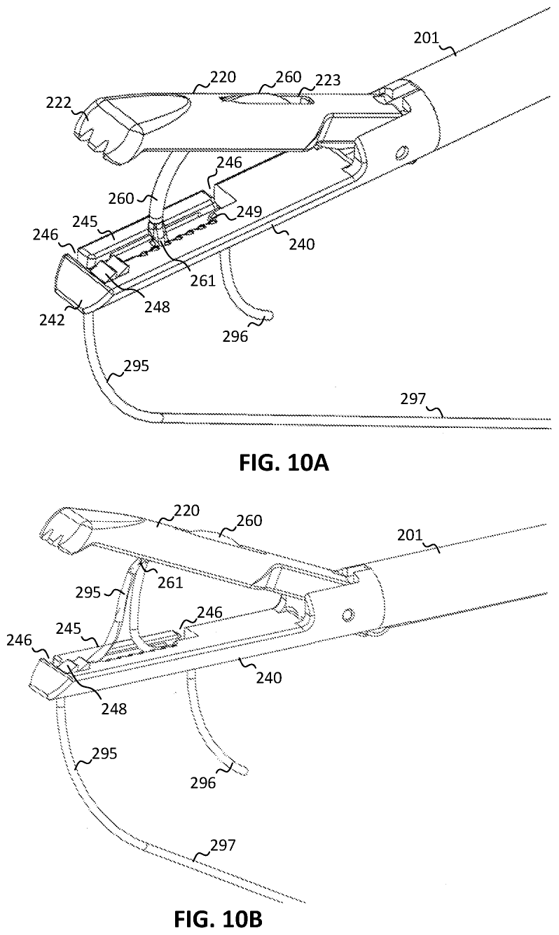

As shown in FIGS. 8, 10A-10B, lower jaw 240 may have internal teeth 248 which serve to help retain tissue held between the upper jaw 220 and lower jaw 240. Internal teeth 248 have an inclined surface facing the distal end 241 of the lower jaw 240 and a relatively vertical wall facing away from the distal end 241 of the lower jaw 240. This shape is akin to backward facing shark teeth in retaining material held within the jaws 220/240 and minimizing or preventing slip of tissue material once the jaws 220/240 are gripping the material. Thus, the external teeth 222 on the upper jaw 220 and the internal teeth 248 on the lower jaw 240 may work together to firmly hold tissue material within the mouth of the jaw while the suture pushing mechanism is used to push suture through the tissue material, as will be described in more detail below.

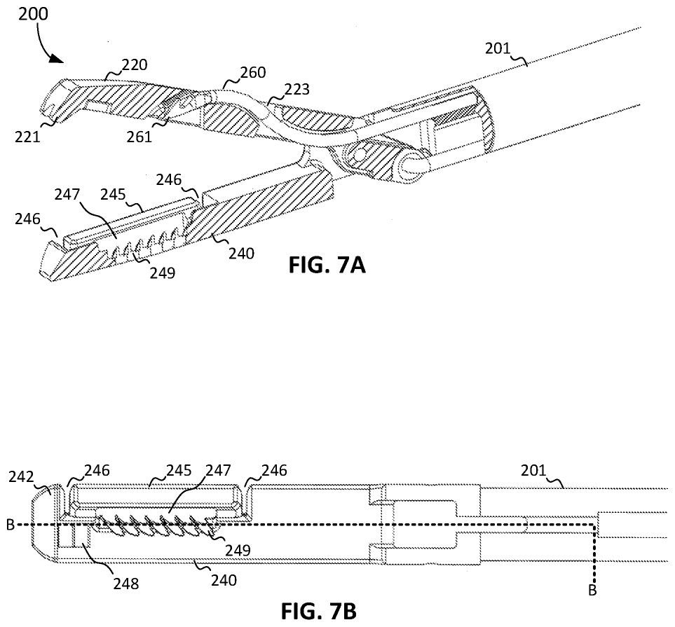

FIG. 7A shows a perspective view with partial cross sectional cut along line B-B shown in the top view illustration of FIG. 7B. It should be noted that FIG. 7B does not have the top jaw portion 220 while FIG. 7A does include the top jaw portion 220. As shown in FIG. 7A, top jaw 220 includes an opening 223 which allows a needle 260 body to traverse therethrough. The needle 260 includes a substantial portion which is housed within the elongated shaft 201 of the device 200, and terminates at a distally positioned suture engagement end 261.

V-shaped asymmetric trough 246 is formed in the bottom jaw 240 and is used to accommodate a suture 295 before it is pulled through tissue. The top down view of the trough 246 may be any shape, including oval, rectangular, circular, etc., as long as it performs the function as shown and described herein. In this particular example, the trough 246 may include three portions, including an elongated central portion which extends along the longitudinal axis of the shaft 201, and the lower jaw 240, and two connecting portions which are perpendicular to the elongated portion. The elongated central portion of the asymmetric trough 246 also includes an opening through which the tip 262 of the needle 260 projects therethrough in order to capture the suture 295, as will be described in more detail below. The trough 246 also includes two connecting portions which extend out from a side of the lower portion 240, as shown in FIG. 7B. A central wall 245 serves to form an internal wall of the trough 246. A suture is routed through the three portions of the trough 246, as will be described in more detail below.

FIG. 8 shows a strand of suture 295 placed within the trough 246 of bottom jaw 240. The strand of suture 295 is wrapped around the vertical wall 245 and pulled so that the two ends 296/297 of the suture 295 extend out beyond the edges of lower jaw 240. A proximal end 296 of the suture 295 is positioned closer to the shaft 201, and a distal end 297 is positioned further from the shaft 201 and closer to the end 241 of the lower jaw 240. FIG. 8 illustrates the position of the suture 295 within the trough 246 of the device 200 directly before use of the device 200. It should be noted that although the trough 246 in the exemplary embodiment has been shown in the figures as having a specific configuration, the same technique may be used if the trough holding the suture was oriented 90 degrees (or other angle) to what is shown in the drawings. In other words, instead of the central portion of the trough 246 being longitudinal across the lower jaw 240, it is transverse.

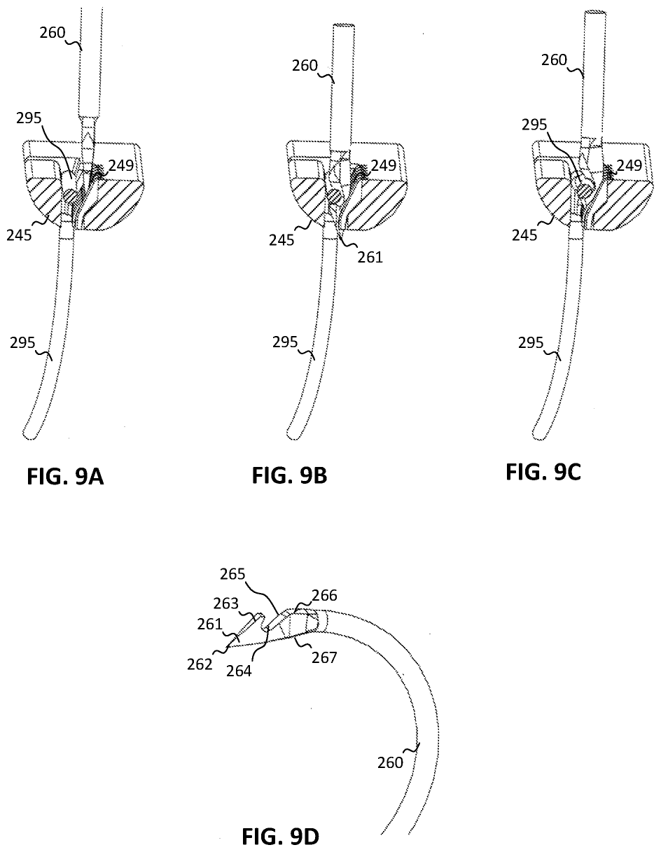

FIG. 9D shows a detailed view of a needle 260 used in the exemplary embodiment presented in FIGS. 6-10 of the present disclosure. Needle 260 includes an elongated shaft with a suture engaging portion 261 at its distal end. The suture engaging portion 261 includes a pointed or sharpened tip 262 and two sides, a top side 266 and a bottom side 267. A straight edge bottom side 267 includes a tapered edge. A top side 266 has a flattened, square edge. Three edges are formed between the top side 266 and bottom side 267. The top side 266 has two edges and the bottom side 267 has one edge. The angle formed between the bottom side 267 edge and top side 266 edges may be, for example, between 10 to 60 degrees. In particular, the angle formed may be between 30-45 degrees. One exemplary angle edge is 30 degrees. Another exemplary angle edge is 45 degrees. The angled edge of the suture engaging portion 261 along with the angled grooves 249 within the trough 247 serve to twist the suture engaging portion 261, which serves a function of engaging a strand of suture, as will be described in detail below.

The suture engaging portion 261 includes a hook portion 263/264/265 in the shape of a crochet hook. The hook portion 263/264/265 includes a first edge 263, a concave trough 264, and a top sloped edge 265 that leads into the top side 266 of the suture engaging portion 261. The concave trough 264 accommodates a strand of suture 295 so it should be sized and textured to hold various sized sutures without damaging them.

FIGS. 9A-9C show the interaction of the suture engaging portion 261 of the needle 260 with the trough 246. As shown in FIG. 9A, as the suture engaging portion 261 enters the trough 246, the hook portion 263/264/265 makes contact with one of a plurality of angled grooves 249 positioned within the sloped sidewall of the concave trough 247. The tapered shape of the suture engaging portion 261 adapts it to twist so that the needle tip 262 angles underneath the strand of suture 295, as shown in FIG. 9B. The strand of suture 295 is then positioned within the concave trough 264. As shown in FIG. 9C, the strand 295 is positioned within the concave trough 264 as the needle tip 262 is being pulled back up so that the strand of suture 295 is fully controlled by suture engaging portion 261.

The external shape of the suture engaging portion 261 along with the shape of the plurality of angled grooves 249 allows an angled twist of the suture engaging portion 261 as it comes into contact with the angled grooves 249 of the trough 247, thereby positioning the hook portion 263/264/265 underneath the strand of suture 295 so that the strand of suture 295 is cradled and pulled up, as will be described in more detail below. The plurality of angled grooves 249 allows for a wider range of allowable surface contact area of the tip 262 of the needle as it contacts the trough 247. Further, since once the tip 262 of the suture engaging portion 261 comes into contact with one of the plurality of angled grooves 249, the tip 262 is then captured within the groove 249 and forced to twist without sliding further along the length of the groove 247, thereby minimizing movement of the needle 260 within the target tissue before the suture 295 is engaged. Alternatively, it may be possible that the angled grooves 249 induce enough of a twist motion that even a flat needle tip would rotate upon contact to pick up a suture 295.

FIG. 10A shows a perspective view of the device 100 as the suture engaging portion 261 is engaged with the strand of suture 295, the detail of which is shown in FIG. 9C. Once the concave trough 264 of the needle tip 261 has engaged the strand of suture 295, the needle shaft 260 is pulled back so that the needle 260 retracts back into its original starting position, as shown in FIG. 10B. The movement of the suture engaging portion 261 between FIG. 10A and FIG. 10B results in the pulling up of the strand of suture 295 through any tissue material that is held in place between the upper jaw 220 and lower jaw 240.

In use, the device 200 starts in a closed jaw position, such as those shown in FIGS. 6A-6D. Once a given tissue is desired to be repaired, the jaws 220/240 are opened, revealing an internal trough 246. A strand of suture 295 is then positioned within the trough 246 so that the ends 296/297 of the suture 295 extend out from the edge of the lower jaw 240, as shown in FIG. 8. The open jaws 220/240 are then positioned about a target tissue which needs to be repaired. The external teeth 222 and internal teeth 248 service to maintain the position of the target tissue (not shown) with respect to the jaws 220/240. Once the target tissue (not shown) is positioned and held within the open jaws 220/240, as shown in FIG. 8, the needle 260 is then pushed through the target tissue. The suture engaging portion 261 is pushed through the tissue until it comes into contact with one of the angled grooves 249 positioned on the sloped wall 247 of the bottom jaw 240, as shown in FIG. 9A. The suture engaging portion 261 then twists and extends underneath the strand of suture 295 that was positioned in trough 246 until the hook portion 263/264/265 slips underneath the strand of suture 295, as shown in FIGS. 9B and 10A. Once the strand of suture 295 is cradled within the suture trough 264, the needle 260 is pulled back up through the target tissue, bringing with it the strand of suture 295, as shown in FIGS. 9C and 10B. At this point, the strand of suture 295 has been pulled through the tissue and may be used in creating various stitches as desired.

FIGS. 11-15 illustrate a suture passer device according to a third exemplary embodiment of the present subject disclosure. The device 300 includes an elongated shaft body 301 connecting to a pair of jaws, including an upper jaw 320 and a lower jaw 340. The upper jaw 320 and lower jaw 340 move relative to each other about a rotation point 302, which may be a hinge or other similar mechanism. Upper jaw 320 has a distal end 321 and lower jaw 340 has a distal end 341. The upper jaw distal end 321 is adapted to fit over the lower jaw distal end 341. However, the opposite is also possible in that the lower jaw distal end 341 may be adapted to fit over the upper jaw distal end 321.

Upper jaw distal end 321 may have one or more external teeth 322 which serve to grab unto tissue held within the pair of jaws 320/340. The lower jaw distal end 341 may have a generally flat and inclined surface to accommodate an opposite side of the tissue which is being held by the external teeth 322. Optionally, external teeth 322 may be present on both the upper jaw distal end 321 and lower jaw distal end 341. Also possible is that the lower jaw distal end 341 has external teeth 322, or neither jaw 320/340 has any teeth at all.

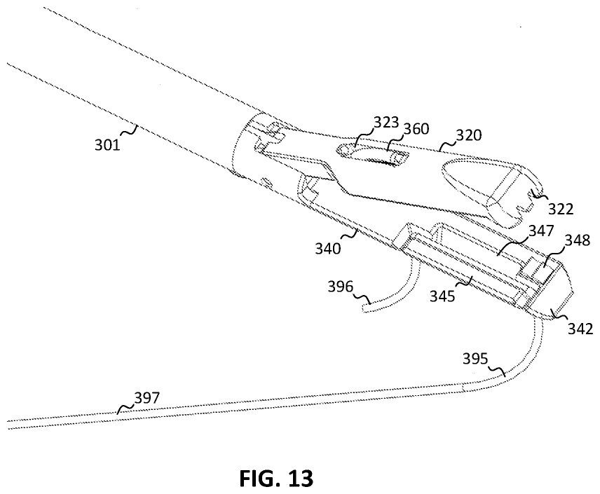

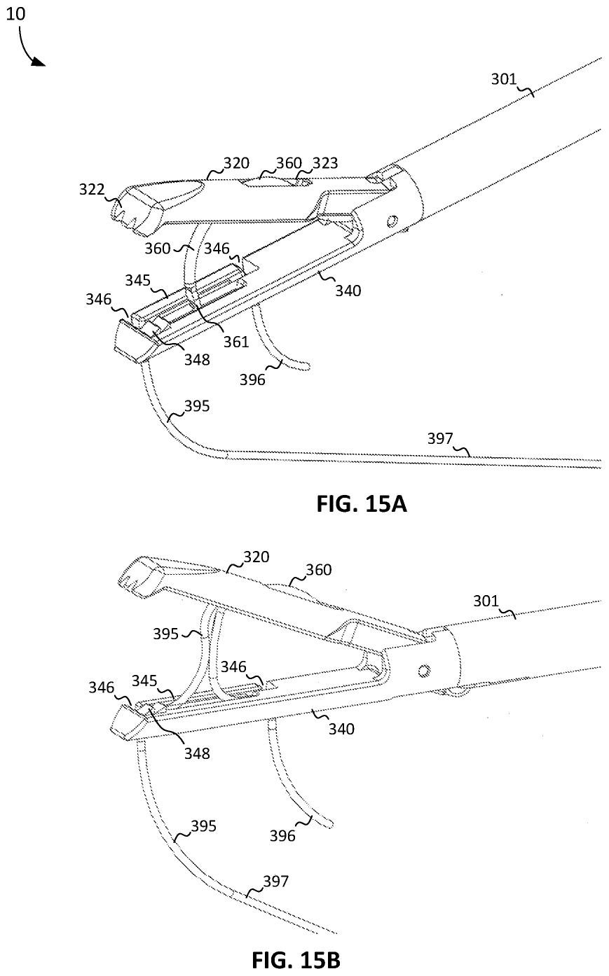

As shown in FIGS. 13, 15A-15B, lower jaw 340 may have internal teeth 348 which serve to help retain tissue held between the upper jaw 320 and lower jaw 340. Internal teeth 348 have an inclined surface facing the distal end 341 of the lower jaw 340 and a relatively vertical wall facing away from the distal end 341 of the lower jaw 340. This shape is akin to backward facing shark teeth in retaining material held within the jaws 320/340 and minimizing or preventing slip of tissue material once the jaws 320/340 are gripping the material. Thus, the external teeth 322 on the upper jaw 320 and the internal teeth 348 on the lower jaw 340 may work together to firmly hold tissue material within the mouth of the jaw while the suture pushing mechanism is used to push suture through the tissue material, as will be described in more detail below.

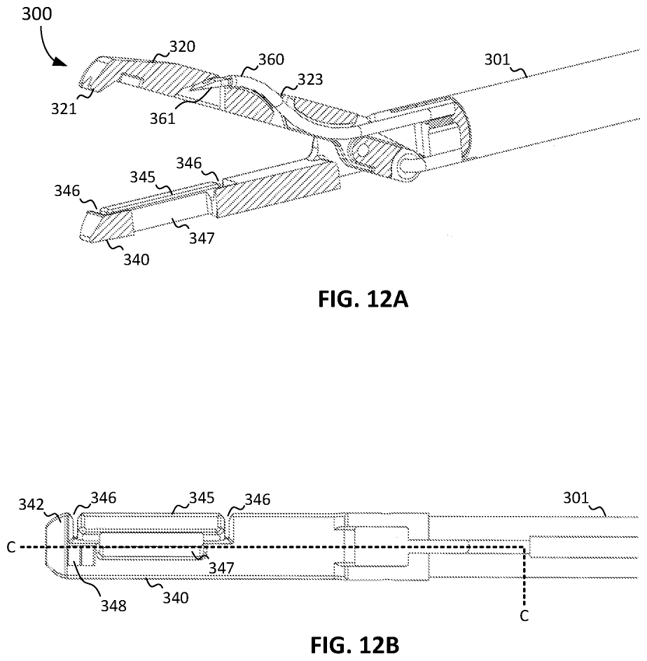

FIG. 12A shows a perspective view with partial cross sectional cut along line C-C shown in the top view illustration of FIG. 12B. It should be noted that FIG. 12B does not have the top jaw portion 320 while FIG. 12A does include the top jaw portion 320. As shown in FIG. 12A, top jaw 320 includes an opening 323 which allows a needle 360 body to traverse therethrough. The needle 360 includes a substantial portion which is housed within the elongated shaft 301 of the device 300, and terminates at a distally positioned suture engagement end 361. V-shaped asymmetric trough 346 is formed in the bottom jaw 340 and is used to accommodate a suture before it is pulled through tissue. The top down view of the trough 346 may be any shape, including oval, rectangular, circular, etc., as long as it performs the function as shown and described herein. In this particular example, the trough 346 may include three portions, including an elongated central portion which extends along the longitudinal axis of the shaft 301, and the lower jaw 340, and two connecting portions which are perpendicular to the elongated portion. The elongated central portion of the asymmetric trough 346 also includes an opening through which the tip 362 of the needle 360 projects therethrough in order to capture the suture 395, as will be described in more detail below. The trough 346 also includes two connecting portions which extend out from a side of the lower portion 340, as shown in FIG. 12B, and two connecting portions which are perpendicular to the elongated portion. A central wall 345 serves to form an internal wall of the trough 346. A suture is routed through the three portions of the trough 346, as will be described in more detail below.

FIG. 13 shows a strand of suture 395 placed within the trough 346 of bottom jaw 340. The strand of suture 395 is wrapped around the vertical wall 345 and pulled so that the two ends 396/397 of the suture 395 extend out beyond the edges of lower jaw 340. A proximal end 396 of the suture 395 is positioned closer to the shaft 301, and a distal end 397 is positioned further from the shaft 301 and closer to the end 341 of the lower jaw 340. FIG. 13 illustrates the position of the suture 395 within the trough 346 of the device 300 directly before use of the device 300. It should be noted that although the trough 346 in the exemplary embodiment has been shown in the figures as having a specific configuration, the same technique may be used if the trough holding the suture was oriented 90 degrees (or other angle) to what is shown in the drawings. In other words, instead of the central portion of the trough 346 being longitudinal across the lower jaw 340, it is transverse.

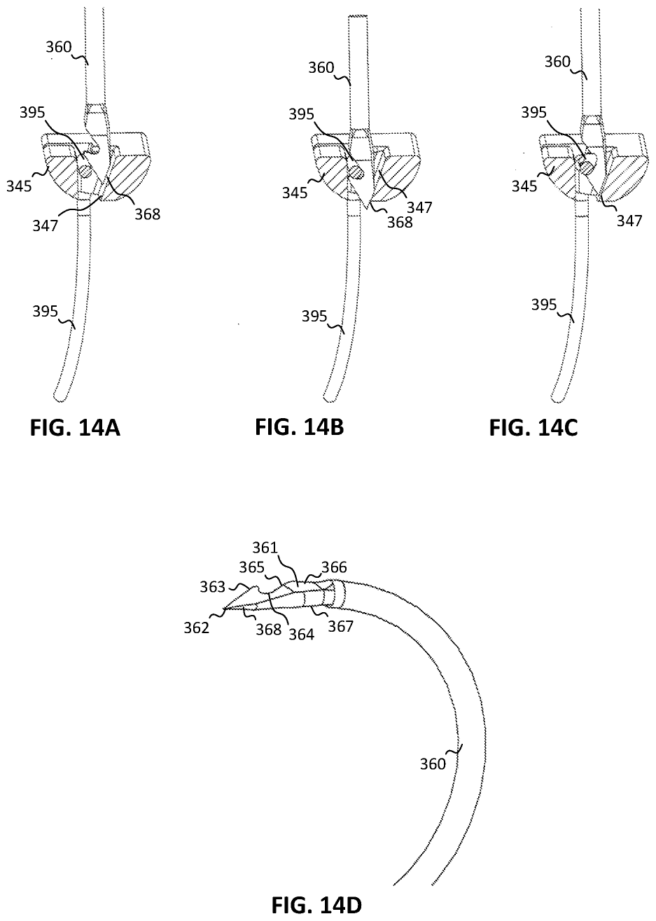

FIG. 14D shows a detailed view of a needle 360 used in the exemplary embodiment presented in FIGS. 11-15 of the present disclosure. Needle 360 includes an elongated shaft with a suture engaging portion 361 at its distal end. The suture engaging portion 361 includes a pointed or sharpened tip 362 and two sides, a top side 366 and a bottom side 367. A straight edge top side 366 includes a tapered edge. A bottom side 367 has a flattened, square edge. Three edges are formed between the top side 366 and bottom side 367. The bottom side 367 has two edges and the top side 366 has one edge. The angle formed between the top side 366 edge and bottom side 367 edges may be, for example, between 10-60 degrees. In particular, the angle formed may be between 30-45 degrees. One exemplary angle edge is 30 degrees. Another exemplary angle edge is 45 degrees. A sloped guiding edge 368 is positioned on the distal end of the bottom side 367. The guiding edge 368 is designed to engage with and slide along the trough 347, as will be described in detail below. In this particular embodiment, the particular angles/shapes of the suture engaging portion 361 could be variable, including round, flat or tapered, with a variable 10-60 degree angle. In any shape of configuration, the needle will deflect off the asymmetric trough and translate toward the side, slightly off axis.

The suture engaging portion 361 includes a hook portion 363/364/365 in the shape of a crochet hook. The hook portion 363/364/365 includes a first edge 363, a concave trough 364, and a top sloped edge 365 that leads into the top side 366 of the suture engaging portion 361. The concave trough 364 accommodates a strand of suture 395 so it should be sized and textured to hold various sized sutures without damaging them.

FIGS. 14A-14C show the interaction of the suture engaging portion 361 of the needle 360 with the trough 346. As shown in FIG. 14A, as the suture engaging portion 361 enters the trough 347, the guiding edge 368 makes contact with the sloped sidewall of the concave trough 347. The sloped shape of the guiding edge 368 adapts it to slide so that the first edge 363 slides underneath the strand of suture 395, as shown in FIG. 14B. The strand of suture 395 is then positioned within the concave trough 364. As shown in FIG. 14C, the strand 395 is positioned within the concave trough 364 as the needle tip 362 being pulled back up so that the strand of suture 395 is fully controlled by suture engaging portion 361.

The external shape of the suture engaging portion 361, and in particular the guiding edge 368, allows a sliding motion of the suture engaging portion 361 as it comes into contact with the angled wall of the trough 347, thereby positioning the hook portion 363/364/365 underneath the strand of suture 395 so that the strand of suture 395 is cradled and pulled up, as will be described in more detail below.

FIG. 15A shows a perspective view of the device 300 as the suture engaging portion 361 is engaged with the strand of suture 395, the detail of which is shown in FIG. 14C. Once the concave trough 364 of the needle tip 361 has engaged the strand of suture 395, the needle shaft 360 is pulled back so that the needle 360 retracts back into its original starting position, as shown in FIG. 15B. The movement of the suture engaging portion 361 between FIG. 15A and FIG. 15B results in the pulling up of the strand of suture 395 through any tissue material that is held in place between the upper jaw 320 and lower jaw 340.

In use, the device 300 starts in a closed jaw position, such as those shown in FIGS. 11A-11D. Once a given tissue is desired to be repaired, the jaws 320/340 are opened, revealing an internal trough 346. A strand of suture 395 is then positioned within the trough 346 so that the ends 396/397 of the suture 395 extend out from the edge of the lower jaw 340, as shown in FIG. 13. The open jaws 320/340 are then positioned about a target tissue which needs to be repaired. The external teeth 322 and internal teeth 348 service to maintain the position of the target tissue with respect to the jaws 320/340. Once the target tissue (not shown) is positioned and held within the open jaws 320/340, as shown in FIG. 13, the needle 360 is then pushed through the target tissue. The suture engaging portion 361 is pushed through the tissue until it comes into contact with the sloped wall 347 of the bottom jaw 340, as shown in FIG. 14A. The suture engaging portion 361 then is guided, through contact with the guiding edge 368 with the sloped wall of the trough 347, and extends underneath the strand of suture 395 that was positioned in trough 346 until the hook portion 363/364/365 slips underneath the strand of suture 395, as shown in FIGS. 14B and 15A. Once the strand of suture 395 is cradled within the suture trough 364, the needle 360 is pulled back up through the target tissue, bringing with it the strand of suture 395, as shown in FIGS. 14C and 15B. At this point, the strand of suture has been pulled through the tissue and may be used in creating various stitches as desired.

It should be noted that the overall shape of the suture engaging portion 361 shown in FIGS. 11-15 is similar to the overall shape of the suture engaging portions 161 and 261 shown in FIGS. 1-5, and FIGS. 6-10, respectively, but for a few differences. See FIGS. 4D, 9D, and 14D for configuration of suture engaging portions 161, 261, and 361, respectively. The suture engaging portions 161 and 261 are substantially identical in that the lower portions 167 and 267 have the sharp edges and the top portion 166 and 266 have the square edges. In contrast, the suture engaging portion 361 has a sharp edge on its top side 366 and a square edge on its bottom side 367. Further, suture engaging portions 161 and 261 both contact the troughs 147 and 247, which causes them to spin underneath a strand of suture. In contrast, suture engaging portion 361 does not spin but contacts the trough 347 via an additional guiding edge 368 positioned on its lower side 367. The guiding side 368 slides along the trough 347 (without spinning) so that the suture engaging portion 361 slides underneath and engages a strand of suture 395.

FIGS. 16-20 illustrate a suture passer device according to a fourth exemplary embodiment of the present subject disclosure. The device 400 includes an elongated shaft body 401 connecting to a pair of jaws, including an upper jaw 420 and a lower jaw 440. The upper jaw 420 and lower jaw 440 move relative to each other about a rotation point 402, which may be a hinge or other similar mechanism. Upper jaw 420 has a distal end 421 and lower jaw 440 has a distal end 441. The upper jaw distal end 421 is adapted to fit over the lower jaw distal end 441. However, the opposite is also possible in that the lower jaw distal end 441 may be adapted to fit over the upper jaw distal end 421.

Upper jaw distal end 421 may have one or more external teeth 422 which serve to grab unto tissue held within the pair of jaws 420/440. The lower jaw distal end 441 may have a generally flat and inclined surface to accommodate an opposite side of the tissue which is being held by the external teeth 422. Optionally, external teeth 422 may be present on both the upper jaw distal end 421 and lower jaw distal end 441. Also possible is that the lower jaw distal end 441 has external teeth 422, or neither jaw 420/440 has any teeth at all.

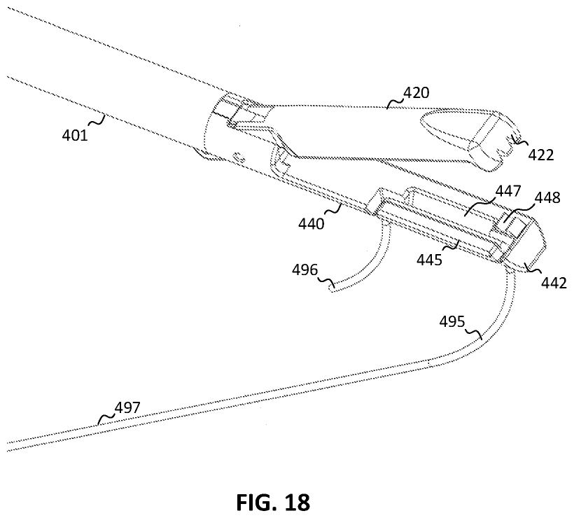

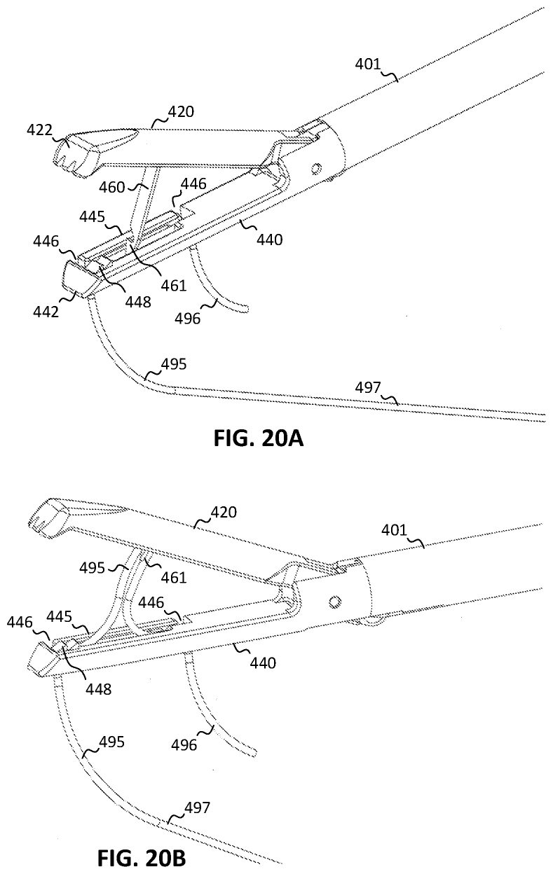

As shown in FIGS. 18, 20A-20B, lower jaw 440 may have internal teeth 448 which serve to help retain tissue held between the upper jaw 420 and lower jaw 440. Internal teeth 448 have an inclined surface facing the distal end 441 of the lower jaw 440 and a relatively vertical wall facing away from the distal end 441 of the lower jaw 440. This shape is akin to backward facing shark teeth in retaining material held within the jaws 420/440 and minimizing or preventing slip of tissue material once the jaws 420/440 are gripping the material. Thus, the external teeth 422 on the upper jaw 420 and the internal teeth 448 on the lower jaw 440 may work together to firmly hold tissue material within the mouth of the jaw while the suture pulling mechanism is used to pull suture through the tissue material, as will be described in more detail below.

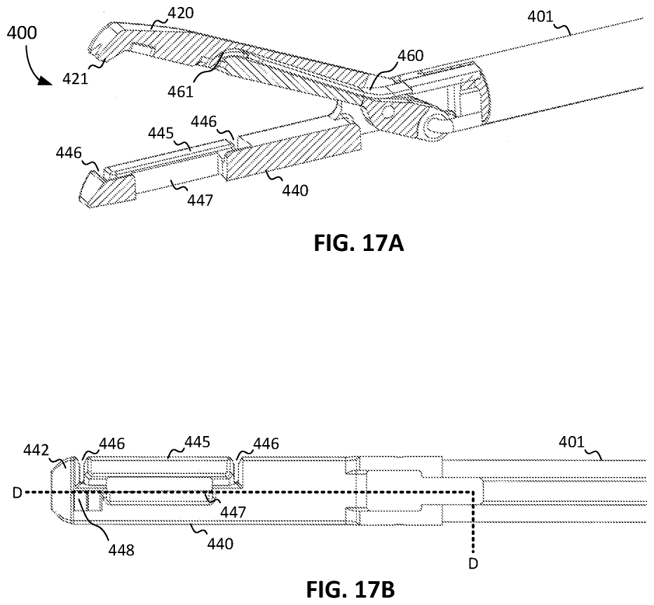

FIG. 17A shows a perspective view with partial cross sectional cut along line D-D shown in the top view illustration of FIG. 17B. It should be noted that FIG. 17B does not have the top jaw portion 420 while FIG. 17A does include the top jaw portion 420. As shown in FIG. 17A, top jaw 420 includes an opening 423 which allows a needle 460 body to traverse therethrough. The needle 460 includes a substantial portion which is housed within the elongated shaft 401 of the device 400, and terminates at a distally positioned suture engagement end 461.

V-shaped asymmetric trough 446 is formed in the bottom jaw 440 and is used to accommodate a suture before it is pulled through tissue. The top down view of the trough 446 may be any shape, including oval, rectangular, circular, etc., as long as it performs the function as shown and described herein. In this particular example, the trough 446 may include three portions, including an elongated central portion which extends along the longitudinal axis of the shaft 401, and the lower jaw 440, and two connecting portions which are perpendicular to the elongated portion. The elongated central portion of the asymmetric trough 446 also includes an opening through which the tip 462 of the needle 460 projects therethrough in order to capture the suture 495, as will be described in more detail below. The trough 446 also includes two connecting portions which extend out from a side of the lower portion 440, as shown in FIG. 17B. A central wall 445 serves to form an internal wall of the trough 446. A suture is routed through the three portions of the trough 446, as will be described in more detail below.

FIG. 18 shows a strand of suture 495 placed within the trough 446 of bottom jaw 440. The strand of suture 495 is wrapped around the vertical wall 445 and pulled so that the two ends 496/497 of the suture 495 extend out beyond the edges of lower jaw 440. A proximal end 496 of the suture 495 is positioned closer to the shaft 401, and a distal end 497 is positioned further from the shaft 401 and closer to the end 441 of the lower jaw 440. FIG. 18 illustrates the position of the suture 495 within the trough 446 of the device 400 directly before use of the device 400. It should be noted that although the trough 446 in the exemplary embodiment has been shown in the figures as having a specific configuration, the same technique may be used if the trough holding the suture was oriented 90 degrees (or other angle) to what is shown in the drawings. In other words, instead of the central portion of the trough 446 being longitudinal across the lower jaw 440, it is transverse.

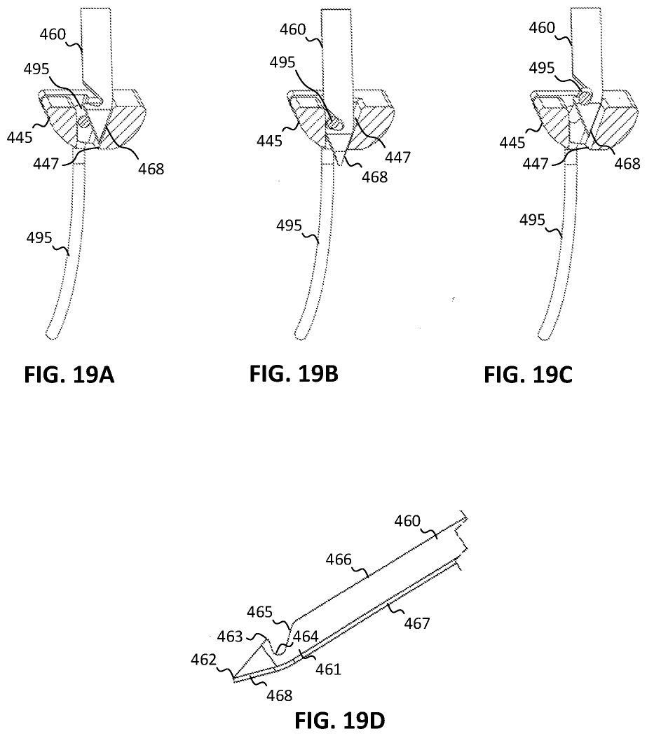

FIG. 19D shows a detailed view of a needle 460 used in the exemplary embodiment presented in FIGS. 16-20 of the present disclosure. Needle 460 is substantially flat and includes an elongated shaft with a suture engaging portion 461 at its distal end. The suture engaging portion 461 includes a pointed or sharpened tip 462 and two sides, a bottom side 467 and a top side 466. A sloped guiding edge 468 is positioned on the distal end of the bottom side 467. The guiding edge 468 is designed to engage with and slide along the trough 447, as will be described in detail below.

The suture engaging portion 461 includes a hook portion 463/464/465 in the shape of a crochet hook. The hook portion 463/464/465 includes a first edge 463, a concave trough 464, and a top sloped edge 465 that leads into the top side 466 of the suture engaging portion 461. The concave trough 464 accommodates a strand of suture 495 so it should be sized and textured to hold various sized sutures without damaging them.

FIGS. 19A-19C show the interaction of the suture engaging portion 461 of the needle 460 with the trough 446. As shown in FIG. 19A, as the suture engaging portion 461 enters the trough 447, the guiding edge 468 makes contact with the sloped sidewall of the concave trough 447. The sloped shape of the guiding edge 468 adapts it to slide so that the first edge 463 slides underneath the strand of suture 495, as shown in FIG. 14B. The strand of suture 495 is then positioned within the concave trough 464. As shown in FIG. 19C, the strand 495 is positioned within the concave trough 464 as the needle tip 462 is being pulled back up so that the strand of suture 495 is fully controlled by suture engaging portion 461.

The external shape of the suture engaging portion 461, and in particular the guiding edge 468, allows a sliding motion of the suture engaging portion 461 as it comes into contact with the angled wall of the trough 447, thereby positioning the hook portion 463/464/465 underneath the strand of suture 495 so that the strand of suture 495 is cradled and pulled up, as will be described in more detail below.

FIG. 20A shows a perspective view of the device 400 as the suture engaging portion 461 is engaged with the strand of suture 495, the detail of which is shown in FIG. 19C. Once the concave trough 464 of the suture engaging portion 461 has engaged the strand of suture 495, the needle shaft 460 is pulled back so that the needle 460 retracts back into its original starting position, as shown in FIG. 20B. The movement of the suture engaging portion 461 between FIG. 20A and FIG. 20B results in the pulling up of the strand of suture 495 through any tissue material that is held in place between the upper jaw 420 and lower jaw 440.

In use, the device 400 starts in a closed jaw position, such as those shown in FIGS. 16A-16D. Once a given tissue is desired to be repaired, the jaws 420/440 are opened, revealing an internal trough 446. A strand of suture 495 is then positioned within the trough 446 so that the ends 496/497 of the suture 495 extend out from the edge of the lower jaw 440, as shown in FIG. 18. The open jaws 420/440 are then positioned about a target tissue which needs to be repaired. The external teeth 422 and internal teeth 448 service to maintain the position of the target tissue (not shown) with respect to the jaws 420/440. Once the target tissue (not shown) is positioned and held within the open jaws 420/440, as shown in FIG. 18, the needle 460 is then pushed through the target tissue. The suture engaging portion 461 is pushed through the tissue until it comes into contact with the sloped wall 447 of the bottom jaw 440, as shown in FIG. 19A. The suture engaging portion 461 then is guided, through contact with the guiding edge 468 with the sloped wall of the trough 447, and extends underneath the strand of suture 495 that was positioned in trough 446 until the hook portion 463/464/465 slips underneath the strand of suture 495, as shown in FIGS. 19B and 20A. Once the strand of suture 495 is cradled within the suture trough 464, the needle 460 is pulled back up through the target tissue, bringing with it the strand of suture 495, as shown in FIGS. 19C and 20B. At this point, the strand of suture has been pulled through the tissue and may be used in creating various stitches as desired.

FIGS. 21-25 illustrate a suture passer device according to a fifth exemplary embodiment of the present subject disclosure. In this fifth exemplary embodiment, the trough is positioned transverse to the longitudinal axis of the bottom jaw, as opposed to the trough being on or parallel to the longitudinal axis of the lower jaw, as shown in FIGS. 1-20. Further, the fifth embodiment shown in FIGS. 21-25 shows a curved crochet needle positioned so that its suture engaging portion is on the inner radius of its movement within the jaws. The needle passes through tissue, encounters a sloped surface in the trough, deflects into a tighter radius of curvature underneath the suture, which is lying in the transversely oriented slot, and picks up the suture on the return. The benefits of having the crochet hook on the inner curve includes, for example, less drag on the suture as it is withdrawn through the tissue, helping to maintain it in the crochet hook. The inner curve would have less friction than the outer curved surface of the needle, and the suture has a shorter distance to be pulled through tissue.

One of the many benefits of the present subject disclosure is that pulling a suture through tissue, back through a hole already created in the tissue by the needle, encounters less resistance than trying to push a needle along with suture through intact tissue. This is particularly evident when the crochet hook is positioned on the inner curve of the needle.

The device 500 includes an elongated shaft body 501 connecting to a pair of jaws, including an upper jaw 520 and a lower jaw 540. The upper jaw 520 and lower jaw 540 move relative to each other about a rotation point 502, which may be a hinge or other similar mechanism. Upper jaw 520 has a distal end 521 and lower jaw 540 has a distal end 541. The upper jaw distal end 521 is adapted to fit over the lower jaw distal end 541. However, the opposite is also possible in that the lower jaw distal end 541 may be adapted to fit over the upper jaw distal end 521.

Upper jaw distal end 521 may have one or more external teeth 522 which serve to grab unto tissue held within the pair of jaws 520/540. The lower jaw distal end 541 may have a generally flat and inclined surface to accommodate an opposite side of the tissue which is being held by the external teeth 522. Optionally, external teeth 522 may be present on both the upper jaw distal end 521 and lower jaw distal end 541. Also possible is that the lower jaw distal end 541 has external teeth 522, or neither jaw 520/540 has any teeth at all.

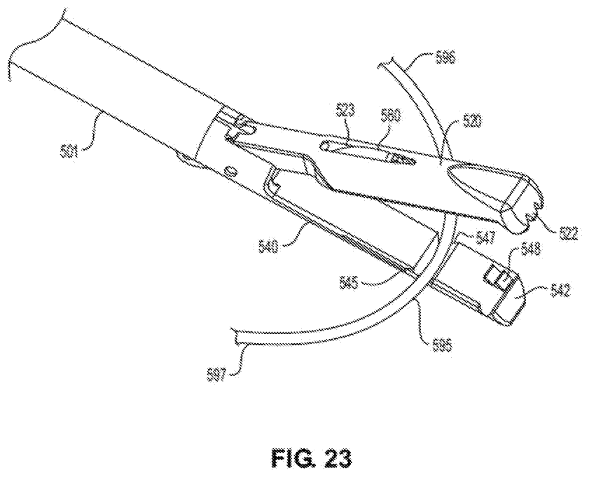

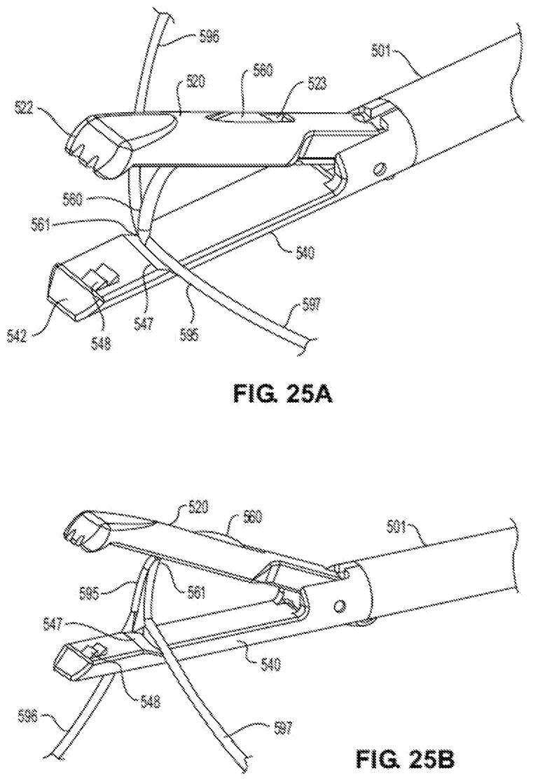

As shown in FIGS. 23, 25A-25B, lower jaw 540 may have internal teeth 548 which serve to help retain tissue held between the upper jaw 520 and lower jaw 540. Internal teeth 548 have an inclined surface facing the distal end 541 of the lower jaw 540 and a relatively vertical wall facing away from the distal end 541 of the lower jaw 540. This shape is akin to backward facing shark teeth in retaining material held within the jaws 520/540 and minimizing or preventing slip of tissue material once the jaws 520/540 are gripping the material. Thus, the external teeth 522 on the upper jaw 520 and the internal teeth 548 on the lower jaw 540 may work together to firmly hold tissue material within the mouth of the jaw while the suture pushing mechanism is used to push suture through the tissue material, as will be described in more detail below.

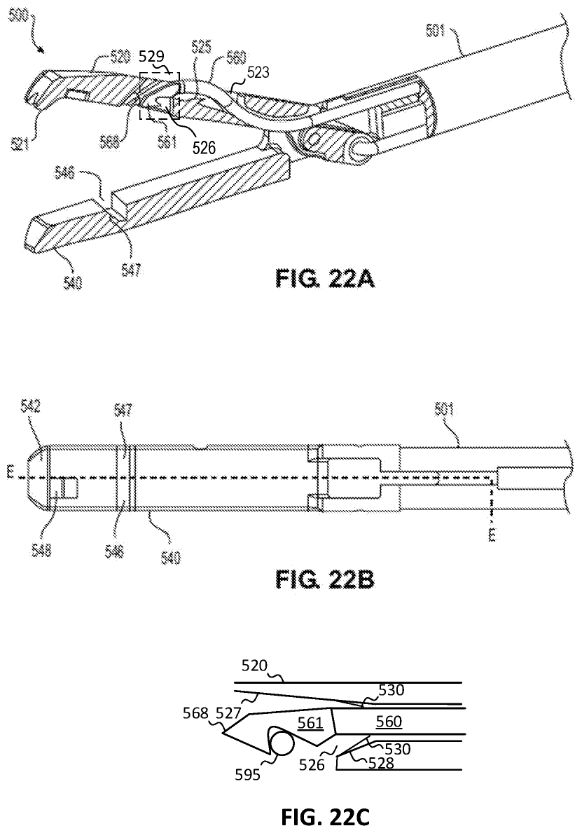

FIG. 22A shows a perspective view with partial cross sectional cut along line E-E shown in the top view illustration of FIG. 22B. It should be noted that FIG. 22B does not have the top jaw portion 520 while FIG. 22A does include the top jaw portion 520. As shown in FIG. 22A, top jaw 520 includes an opening 523 which allows a needle 560 body to traverse therethrough. The needle 560 includes a substantial portion which is housed within the elongated shaft 501 of the device 500, and terminates at a distally positioned suture engagement end 561. A catch hook 525 positioned within the upper jaw 520 is used to capture a pulled suture strand 595 as will be described in more detail below.

V-shaped asymmetric trough 546 is formed in the bottom jaw 540 and is used to accommodate a suture before it is pulled through tissue. The top down view of the trough 546 may be any shape, including oval, rectangular, circular, etc., as long as it performs the function as shown and described herein. In this particular example, the trough 546 includes an elongated portion which extends transverse to the longitudinal axis of the shaft 501, and the lower jaw 540. The elongated portion of the asymmetric trough 546 can also include an opening through which the tip 562 of the needle 560 projects therethrough in order to capture the suture 595, as will be described in more detail below. The asymmetric trough 546 includes a distal wall 547 and a proximal wall 545. The distal wall 547 is sloped to aid the tip 562 of the needle 560 to a position underneath the suture. The proximal wall 545 is closer to a vertical position, such as a 90 degree angle, in order to allow the suture to be anchored in position during use. A suture is routed through the trough 546 to position it in place for use, as will be described in more detail below.

FIG. 23 shows a strand of suture 595 placed within the trough 546 of bottom jaw 540. The strand of suture 595 is wrapped around the proximal wall 545 and pulled so that the two ends 596/597 of the suture 595 extend out beyond the edges of lower jaw 540.

FIG. 23 illustrates the position of the suture 595 within the trough 546 of the device 500 directly before use of the device 500. It should be noted that although the trough 546 in the exemplary embodiment has been shown in the figures as having a specific configuration, the same technique may be used if the trough holding the suture was oriented at an angle to what is shown in the drawings. FIGS. 1-20 showed the needle-suture engaging portion of the trough being parallel (or zero degrees) to the longitudinal axis of the lower jaw. FIGS. 21-25 show the trough being 90 degrees to the longitudinal axis of the lower jaw. The trough can be positioned at any angle with respect to the lower jaw. In other words, the trough can be positioned at 0-90 degrees with respect to the longitudinal axis of the lower jaw.

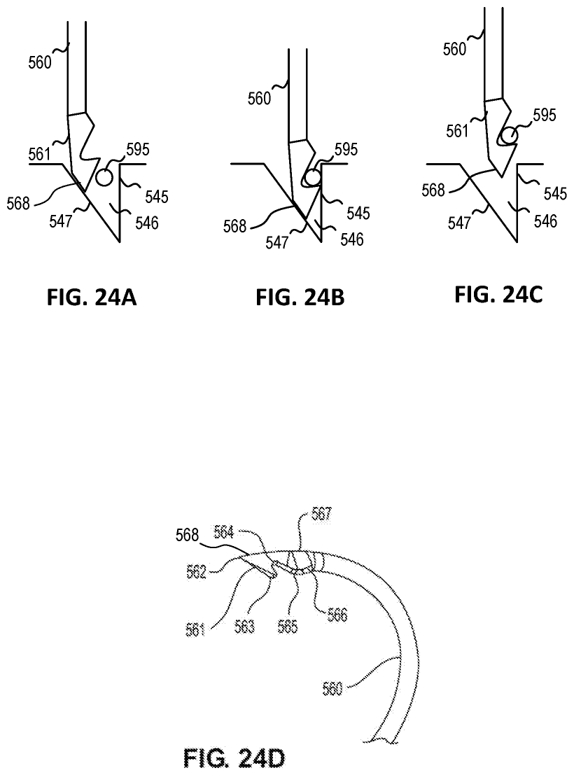

FIG. 24D shows a detailed view of a needle 560 used in the exemplary embodiment presented in FIGS. 21-25 of the present disclosure. Needle 560 includes an elongated shaft with a suture engaging portion 561 at its distal end. The suture engaging portion 561 includes a pointed or sharpened tip 562 and two sides, a top side 566 and a bottom side 567. A straight edge bottom side 566 includes a flattened, square edge. A top side 567 has a tapered edge. Three edges are formed between the top side 567 and bottom side 566. The bottom side 566 has two edges and the top side 567 has one edge. The angle formed between the top side 567 edge and bottom side 566 edges may be, for example, between 10-60 degrees. One exemplary angle edge is 30 degrees. Another exemplary angle edge is 45 degrees. A sloped guiding edge (see guiding edge 368 in FIG. 14D) may be positioned on the distal end of the bottom side 567. The guiding edge is designed to engage with and slide along the trough 547, as will be described in detail below. In this particular embodiment, the particular angles/shapes of the suture engaging portion 561 could be variable, including round, flat or tapered, with a variable 10-60 degree angle. In any shape of configuration, the needle will deflect off the asymmetric trough 546 and translate toward the proximal wall 545, slightly off axis.

The suture engaging portion 561 includes a hook portion 563/564/565 in the shape of a crochet hook. The hook portion 563/564/565 includes a first edge 563, a concave trough 564, and a sloped edge 565 that leads into the bottom side 566 of the suture engaging portion 561. The concave trough 564 accommodates a strand of suture 595 so it should be sized and textured to hold various sized sutures without damaging them.

FIGS. 24A-24C show the interaction of the suture engaging portion 561 of the needle 560 with the trough 546. As shown in FIG. 24A, as the suture engaging portion 561 enters the trough 547, the guiding edge 568 makes contact with the sloped sidewall of the concave trough 547. The sloped shape of the guiding edge 568 adapts it to slide so that the first edge 563 slides underneath the strand of suture 595, as shown in FIG. 24B. The strand of suture 595 is then positioned within the concave trough 564. As shown in FIG. 24C, the strand 595 is positioned within the concave trough 564 as the needle tip 562 being pulled back up so that the strand of suture 595 is fully controlled by suture engaging portion 561.

The external shape of the suture engaging portion 561, and in particular the guiding edge 568, allows a sliding motion of the suture engaging portion 561 as it comes into contact with the angled wall of the trough 547, thereby positioning the hook portion 563/564/565 underneath the strand of suture 595 so that the strand of suture 595 is cradled and pulled up, as will be described in more detail below.

FIG. 25A shows a perspective view of the device 500 as the suture engaging portion 561 is engaged with the strand of suture 595, the detail of which is shown in FIG. 24C. Once the concave trough 564 of the needle tip 561 has engaged the strand of suture 595, the needle shaft 560 is pulled back so that the needle 560 retracts back into its original starting position, as shown in FIG. 25B.

A catch mechanism, such as catch hook 525 (See FIG. 22A), is used to capture and transfer the strand of suture 595 from the crochet hook 561 as the needle 560 pulls back into the upper jaw 520. As the needle 560 is withdrawn with the suture in the crochet hook, it moves partially into the tubular or oval housing for the needle in the upper jaw, pinching the strand of suture against the capture hook 525 in the walls of the upper jaw 520 to hold it for withdrawal. Once outside the joint, the needle 561 can then be advanced slightly to release the suture. The movement of the suture engaging portion 561 between FIG. 25A and FIG. 25B results in the pulling up of the strand of suture 595 through any tissue material that is held in place between the upper jaw 520 and lower jaw 540.

FIG. 22C is a side view enlargement of the boxed area 529 from FIG. 22A, and shows another exemplary suture capture mechanism in which the tubular housing within the upper jaw 520 has a funnel or tapered shape 526 which is wider distally to pinch the suture 595 as it is withdrawn with the needle 561 into its resting position. Once out of the joint, the needle 561 can be advanced slightly to release the suture 595. The walls 527/528 of the funnel 526 could have a frictional surface to enhance the pinching. A roughened surface, small teeth, surface bumps or projections, or other surface treatment may be used to cause the surfaces 527/528 of the funnel 526 to be roughened and enhance the capture of the suture 595 within the tube 501 as the needle 561 is withdrawn into the tubular housing, carrying the suture with the crochet hook. Optionally, a closing flap 530 may be positioned in the area 529 shown in FIG. 22C that folds down on top of the suture 595 to pinch it after it has been pulled into the tube 520.

The capture hook 525, capture funnel 526, and closing flap 530 are mutually exclusive features, and each or a combination of one or more may be added to any of the exemplary embodiments shown and described in the present disclosure. Other suture capture mechanisms are also possible.

In use, the device 500 starts in a closed jaw position, such as those shown in FIGS. 21A-21D. Once a given tissue is desired to be repaired, the jaws 520/540 are opened, revealing an internal trough 546. A strand of suture 595 is then positioned within the trough 546 so that the ends 596/597 of the suture 595 extend out from the edge of the lower jaw 540, as shown in FIG. 23. The open jaws 520/540 are then positioned about a target tissue which needs to be repaired. The external teeth 522 and internal teeth 548 service to maintain the position of the target tissue with respect to the jaws 520/540. Once the target tissue (not shown) is positioned and held within the open jaws 520/540, as shown in FIG. 23, the needle 560 is then pushed through the target tissue. The suture engaging portion 561 is pushed through the tissue until it comes into contact with the sloped wall 547 of the bottom jaw 540, as shown in FIG. 24A. The suture engaging portion 561 then is guided, through contact with the guiding edge 568 with the sloped wall of the trough 547, and extends underneath the strand of suture 595 that was positioned in trough 546 until the hook portion 563/564/565 slips underneath the strand of suture 595, as shown in FIGS. 24B and 25A. Once the strand of suture 595 is cradled within the suture trough 564, the needle 560 is pulled back up through the target tissue, bringing with it the strand of suture 595, as shown in FIGS. 24C and 25B. At this point, the strand of suture has been pulled through the tissue and may be used in creating various stitches as desired.

It should be noted that the overall shape of the suture engaging portion 561 shown in FIGS. 21-25 is similar to the overall shape of the suture engaging portions 161 and 261 shown in FIGS. 1-5, and FIGS. 6-10, respectively, but for a few differences. See FIGS. 4D, 9D, and 14D for configuration of suture engaging portions 161, 261, and 361, respectively. The suture engaging portions 161 and 261 are substantially identical in that the lower portions 167 and 267 have the sharp edges and the top portion 166 and 266 have the square edges. In contrast, the suture engaging portion 561 has a sharp edge on its top side 567 and a square edge on its bottom side 566. Further, suture engaging portions 161 and 261 both contact the troughs 147 and 247, which causes them to spin underneath a strand of suture. In contrast, suture engaging portion 561 does not spin but contacts the trough 547 via an additional guiding edge 568 positioned on its lower side 567. The guiding side 568 slides along the trough 547 (without spinning) so that the suture engaging portion 561 slides underneath and engages a strand of suture 595.

FIGS. 26A-26D illustrate a suture passer device according to a sixth exemplary embodiment of the present subject disclosure. This sixth exemplary embodiment is an additional feature which may be combined with any one of the other four exemplary embodiments shown in FIGS. 1-25. In other words, this sixth exemplary embodiment is an “add on” to any of the previous exemplary embodiments described above.

The device 600 includes an elongated shaft body 601 connecting to a pair of jaws, including an upper jaw 620 and a lower jaw 640. The upper jaw 620 and lower jaw 640 move relative to each other about a rotation point 602, which may be a hinge or other similar mechanism. Upper jaw 620 has a distal end 621 and lower jaw 640 has a distal end 641. The upper jaw distal end 621 is adapted to fit over the lower jaw distal end 641. However, the opposite is also possible in that the lower jaw distal end 641 may be adapted to fit over the upper jaw distal end 621. An upper neck portion includes a long neck 628 and a short neck 629. A lower neck portion includes a long neck 648 and a short neck 649. The upper long neck 628, which is adjacent to the upper jaw 620, is positioned atop the lower long neck 648, which is adjacent to the lower jaw 640. The upper short neck 629, which is adjacent to the upper jaw 620, is positioned atop the lower short neck 649, which is adjacent to the lower jaw 640.

The combination of a long neck 628/648 and a short neck 649/649 adjacent the upper 620 and lower 640 jaws serve to create an angled jaw, as shown in FIGS. 26A (top view) and 26C (bottom view). Such angled suture pulled would be beneficial for insertion through the femoral notch for meniscus repair, and other procedures, as appreciated by one having ordinary skill in the art. A related angled suture passer patent application, with U.S. patent application Ser. No. 15/939,934, is incorporated by reference herein in its entirety into this disclosure, and will not be described in any detail here.

The exemplary embodiment shown in FIGS. 26A-26D depict a right angled jaw. However, the angle of the jaw may also be left, upward, downward, or combinations thereof (e.g., upward-right, upward-left, downward-right, downward-left). For sake of simplicity, all of these variations have not been shown, but are within the purview of the present subject disclosure. Each such variation could have certain benefits. For example, an upward angled jaw could help get under the curved femoral condyle while resting on the flatter tibial surface. Other benefits of different angled jaws would be appreciated by one having ordinary skill in the art.

Other variations of the exemplary embodiments presented above are possible. For example, a double pass device may be designed with the present subject disclosure. For example, the present disclosure may be used to design a two-needle device, or a needle that moves into two positions to pull the suture through the tissue at two points to encircle a portion of the tissue.

What has been described above includes examples that provide advantages of the subject disclosure. It is, of course, not possible to describe every conceivable combination of components or methodologies for purposes of describing the subject disclosure, but one of ordinary skill in the art may recognize that many further combinations and permutations of the claimed subject matter are possible. Furthermore, to the extent that the terms “includes,” “has,” “possesses,” and the like are used in the detailed description, claims, appendices and drawings such terms are intended to be inclusive in a manner similar to the term “comprising” as “comprising” is interpreted when employed as a transitional word in a claim.

The illustrations and examples provided herein are for explanatory purposes and are not intended to limit the scope of the appended claims. It will be recognized by those skilled in the art that changes or modifications may be made to the above described embodiment without departing from the broad inventive concepts of the subject disclosure. It is understood therefore that the subject disclosure is not limited to the particular embodiment which is described, but is intended to cover all modifications and changes within the scope and spirit of the subject disclosure.

What is claimed is:

1. A suture pulling device, comprising:
a shaft having a proximal end, a distal end, and a longitudinal axis extending therebetween;
a first jaw member and a second jaw member connected to the shaft distal end, the first jaw member moveable relative to the second jaw member;
a tissue penetrating member positioned substantially within and slidable through the first jaw, the tissue penetrating member including a transverse opening configured to selectively carry a suture;
a suture accommodating trough positioned within the second jaw and adapted to hold a strand of suture;
wherein the tissue penetrating member is slidable between a first and a second position, wherein in the first position the tissue penetrating member is recessed within the first jaw member and wherein in the second position the tissue penetrating member extends through a volume defined within the first jaw and the second jaw so that the transverse opening of the tissue penetrating member engages with the strand of suture positioned in the suture accommodating trough of the second jaw.

2. The suture puffing device of claim 1, wherein the suture accommodating trough is positioned so that two ends of the strand of suture project from a side portion of the second jaw.

3. The suture puffing device of claim 1, wherein the suture accommodating trough is positioned so that two ends of the strand of suture project from opposite sides of the second jaw.

4. The suture puffing device of claim 1, wherein the suture accommodating trough has an asymmetric V-shape.

5. The suture puffing device of claim 1 wherein the suture accommodating trough has angled grooves to engage with the tissue penetrating member and twist it upon engagement.

6. The suture puffing device of claim 1, wherein the strand of suture engaged by the transverse opening is maintained within the volume defined within the first jaw and the second jaw.

7. The suture puffing device of claim 1 wherein the volume defined by the first jaw and the second jaw is adapted to hold a sample tissue to be repaired.

8. The suture puffing device of claim 1, wherein the tissue penetrating member is superelastic and is in a stressed position in the first position within the first jaw, and an unstressed, curved position in the second position when not in the first jaw.

9. The suture puffing device of claim 1, wherein the tissue penetrating member has a distal most end that has three edges.

10. The suture puffing device of claim 9, wherein the tissue penetrating member has a top side with two edges and a bottom side with one edge.

11. The suture puffing device of claim 10, wherein the one edge on the bottom side makes a cross section angle of about 10 to 60 degrees with the two edges on the top side.

12. The suture pulling device of claim 9, wherein the tissue penetrating member has a top side with one edge and a bottom side with two edges.

13. The suture puffing device of claim 12, wherein the transverse opening is positioned on the top side.

14. The suture puffing device of claim 12, wherein the one edge on the top side makes a cross section angle of about 10 to 60 degrees with the two edges on the bottom side.

15. The suture puffing device of claim 12, further comprising a guiding edge on the bottom side, the guiding edge being nonlinear with the bottom side.

16. The suture puffing device of claim 9, wherein the tissue penetrating member is substantially flat and has a top side and a bottom side.

17. The suture puffing device of claim 16, further comprising a guiding edge on the bottom side, the guiding edge being nonlinear with the bottom edge.

18. The suture puffing device of claim 1, wherein a longitudinal axis of the first jaw and the second jaw is angled from a longitudinal axis of the shaft.

19. A suture puffing device, comprising:
a shaft having a proximal end, a distal end, and a longitudinal axis extending therebetween;
a first jaw member and a second jaw member connected to the shaft distal end, the first jaw member moveable relative to the second jaw member;
a tissue penetrating member positioned substantially within and slidable through the first jaw, the tissue penetrating member including a transverse opening configured to selectively carry a suture;
an asymmetric V-shaped suture accommodating trough positioned within the second jaw and adapted to hold a strand of suture, wherein the suture accommodating trough is positioned so that two ends of the strand of suture project from a side portion of the second jaw;
wherein the tissue penetrating member is slidable between a first and a second position, wherein in the first position the tissue penetrating member is recessed within the first jaw member and wherein in the second position the tissue penetrating member extends through a volume defined within the first jaw and the second jaw so that the transverse opening of the tissue penetrating member engages with the strand of suture positioned in the suture accommodating trough of the second jaw.

20. A suture puffing device, comprising:
a shaft having a proximal end, a distal end, and a longitudinal axis extending therebetween;
a first jaw member and a second jaw member connected to the shaft distal end, the first jaw member moveable relative to the second jaw member;
a tissue penetrating member positioned substantially within and slidable through the first jaw, the tissue penetrating member including a transverse opening configured to selectively carry a suture;
an asymmetric V-shaped suture accommodating trough positioned within the second jaw and adapted to hold a strand of suture, wherein the suture accommodating trough is positioned so that two ends of the strand of suture project from opposite sides of the second jaw;
wherein the tissue penetrating member is slidable between a first and a second position, wherein in the first position the tissue penetrating member is recessed within the first jaw member and wherein in the second position the tissue penetrating member extends through a volume defined within the first jaw and the second jaw so that the transverse opening of the tissue penetrating member engages with the strand of suture positioned in the suture accommodating trough of the second jaw.
